SEQUENCE LISTING (42 sequences)

SEQ ID NO: 1 - DNA, 34 bp, Artificial Sequence
  modified_base (1)...(4): um
uuuucgtggcctgattcattccccagcgauuuut

SEQ ID NO: 2 - DNA, 44 bp, Artificial Sequence
  oligonucleotide
uuuuagcgccgtggcctgattcattccccagcgaccagauuuut

SEQ ID NO: 3 - DNA, 54 bp, Artificial Sequence
  oligonucleotide
uuuutgattagcgccgtggcctgattcattccccagcgaccagatgatcuuuut

SEQ ID NO: 4 - DNA, 70 bp, Artificial Sequence
  modified_base (1)...(4): um
uuuugcgcgtcgtgattagcgccgtggcctgattcattccccagcgaccagatgatcacactcgguuuut

SEQ ID NO: 5 - DNA, 34 bp, Artificial Sequence
  modified_base (1)...(4): um
uuuutcgctggggaatgaatcaggccacguuuua

SEQ ID NO: 6 - DNA, 44 bp, Artificial Sequence
  modified_base (1)...(4): um
uuuutctggtcgctggggaatgaatcaggccacggcgctuuuua

SEQ ID NO: 7 - DNA, 54 bp, Artificial Sequence
  modified_base (1)...(4): um
uuuugatcatctggtcgctggggaatgaatcaggccacggcgctaatcauuuua

SEQ ID NO: 8 - DNA, 70 bp, Artificial Sequence
  modified_base (1)...(4): um
uuuuccgagtgtgatcatctggtcgctggggaatgaatcaggccacggcgctaatcacgacgcgcuuuua

SEQ ID NO: 9 - DNA, 21 bp, Artificial Sequence
  oligonucleotide
gatgaagccaatattgaaacc

SEQ ID NO: 10 - DNA, 18 bp, Artificial Sequence
  oligonucleotide
ctggtcttcatccacgcg

SEQ ID NO: 11 - DNA, 44 bp, Artificial Sequence
  modified_base (1)...(4): um
uuuuagcgccgtggcctgatttattccccagcgaccagauuuut

SEQ ID NO: 12 - DNA, 44 bp, Artificial Sequence
  modified_base (1)...(4): um
uuuutctggtcgctggggaataaatcaggccacggcgctuuuua

SEQ ID NO: 13 - DNA, 34 bp, Artificial Sequence
  modified_base (1)...(14): 2′ O-methyl RNA
uuuucguggccugattcatuccccagcgauuuut

SEQ ID NO: 14 - DNA, 34 bp, Artificial Sequence
  modified_base (1)...(14): 2′ O-methyl RNA
uuuuucgcuggggaatgaaucaggccacguuuua

SEQ ID NO: 15 - DNA, 65 bp, Artificial Sequence
  oligonucleotide
acccgagtgtgatcatctggtcgctggggaataaatcaggccacggcgctaatcacgacgcgctg

SEQ ID NO: 16 - DNA, 65 bp, Artificial Sequence
  oligonucleotide
cagcgcgtcgtgattagcgccgtggcctgatttattccccagcgaccagatgatcacactcgggt

SEQ ID NO: 17 - DNA, 57 bp, Artificial Sequence
  oligonucleotide
ggcccaccctcgtgaccaccctgacctaccgcgtgcagtgcttcagccgctaccccg

SEQ ID NO: 18 - DNA, 57 bp, Artificial Sequence
  oligonucleotide
cggggtagcggctgaagcactgcacgcggtaggtcagggtggtcacgagggtgggcc

SEQ ID NO: 19 - DNA, 53 bp, Artificial Sequence
  oligonucleotide
uuuutagcggctgaagcactgcacgccgtaggtcagggtggtcacgagguuuu

SEQ ID NO: 20 - DNA, 53 bp, Artificial Sequence
  oligonucleotide
uuuucctcgtgaccaccctgacctacggcgtgcagtgcttcagccgctauuuu

SEQ ID NO: 21 - DNA, 53 bp, Artificial Sequence
  oligonucleotide
uuuutagcggctgaagcactgcacgcggtaggtcagggtggtcacgagguuuu

SEQ ID NO: 22 - DNA, 53 bp, Artificial Sequence
  oligonucleotide
ggggtagcggctgaagcactgcacgccgtaggtcagggtggtcacgaggtggg

SEQ ID NO: 23 - DNA, 53 bp, Artificial Sequence
  oligonucleotide
ggggtagcggctgaagcactgcacgcggtaggtcagggtggtcacgaggtggg

SEQ ID NO: 24 - DNA, 57 bp, Artificial Sequence
  oligonucleotide
tccgccacaacatcgaggacggcagcgtgtagctcgccgaccactaccagcagaaca

SEQ ID NO: 25 - DNA, 57 bp, Artificial Sequence
  oligonucleotide
tgttctgctggtagtggtcggcgagctacacgctgccgtcctcgatgttgtggcgga

SEQ ID NO: 26 - DNA, 53 bp, Artificial Sequence
  oligonucleotide
uuuutgctggtagtggtcggcgagctgcacgctgccgtcctcgatgttguuuu

SEQ ID NO: 27 - DNA, 53 bp, Artificial Sequence
  oligonucleotide
gttctgctggtagtggtcggcgagctgcacgctgccgtcctcgatgttgtggc

SEQ ID NO: 28 - DNA, 53 bp, Artificial Sequence
  oligonucleotide
gttctgctggtagtggtcggcgagctacacgctgccgtcctcgatgttgtggc

SEQ ID NO: 29 - DNA, 57 bp, Artificial Sequence
  oligonucleotide
cgagtgtgatcatctggtcgctggggaataaatcaggccacggcgctaatcacgacg

SEQ ID NO: 30 - DNA, 57 bp, Artificial Sequence
  oligonucleotide
cgtcgtgattagcgccgtggcctgatttattccccagcgaccagatgatcacactcg

SEQ ID NO: 31 - DNA, 53 bp, Artificial Sequence
  oligonucleotide
uuuutgattagcgccgtggcctgattcattccccagcgaccagatgatcuuuu

SEQ ID NO: 32 - DNA, 53 bp, Artificial Sequence
  oligonucleotide
uuuugatcatctggtcgctggggaatgaatcaggccacggcgctaatcauuuu

SEQ ID NO: 33 - DNA, 53 bp, Artificial Sequence
  oligonucleotide
gucgtgattagcgccgtggcctgattcattccccagcgaccagatgatcacac

SEQ ID NO: 34 - DNA, 53 bp, Artificial Sequence
  oligonucleotide
gtcgtgattagcgccgtggcctgattcattccccagcgaccagatgatcacac

SEQ ID NO: 35 - DNA, 53 bp, Artificial Sequence
  oligonucleotide
gtcgtgattagcgccgtggcctgatttattccccagcgaccagatgatcacac

SEQ ID NO: 36 - protein, 16 aa, Artificial Sequence
  protein fragment of tyrosinase
FMGFNCGNSKFGFGGP

SEQ ID NO: 37 - DNA, 48 bp, Artificial Sequence
  oligonucleotide
ttcatgggtttcaactgcggaaactctaagtttggatttgggggccca

SEQ ID NO: 38 - DNA, 48 bp, Artificial Sequence
  oligonucleotide
tgggcccccaaatccaaacttagagtttccgcagttgaaacccatgaa

SEQ ID NO: 39 - DNA, 33 bp, Artificial Sequence
  oligonucleotide
uuuuaatccaaacttacagtttccgcagtuuuu

SEQ ID NO: 40 - DNA, 43 bp, Artificial Sequence
  oligonucleotide
uuuuccccaaatccaaacttacagtttccgcagttgaaauuuu

SEQ ID NO: 41 - DNA, 52 bp, Artificial Sequence
  oligonucleotide
uuuuggcccccaaatccaaacttacagtttccgcagttgaaacccatguuuu

SEQ ID NO: 42 - DNA, 52 bp, Artificial Sequence
  oligonucleotide
uuuuggcccccaaatccaaacttagagtttccgcagttgaaacccatguuuu

(19) United States
(12) Patent Application Publication
(10) Pub. No.: US 2002/0119570 A1
(43) Pub. Date: Aug. 29, 2002

(54) Targeted gene correction by single-stranded oligodeoxynucleotides

(76) Inventors: Kyonggeun Yoon, Berwyn, PA (US); Olga Igoucheva, Philadelphia, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/962,628

(22) Filed: Sep. 25, 2001

Related U.S. Application Data
(60) Provisional application No. 60/235,226, filed on Sep. 25, 2000.

(51) Int. Cl. C12N 15/87

(52) U.S. Cl. 435/455

(57) ABSTRACT

The present invention relates to using single-stranded oligonucleotides that are designed to specifically change a base in a target nucleic acid sequence. This alteration is maintained, expressed and regulated as the normal endogenous gene.

### BACKGROUND OF THE INVENTION

Targeting an oligonucleotide to a genomic DNA or RNA sequence where an alteration is required will result in the repair of that mutation. The current approaches to this therapeutic nucleic acid repair use triple-forming-oligonucleotide technology (Havre, et al., Proc. Natl. Acad. Sci. USA, 90, 7879-83, 1993; Culver, et al., Nat Biotechnol, 17, 989-993, 1999) and chimeric RNA-DNA oligonucleotide technology (Yoon, et al., Proc. Natl. Acad. Sci. USA 93, 2071-2076, 1996; Cole-Strauss, et al., Science, 273, 1386-1389, 1996; Kren, et al., Heptatogy, 25, 1462-1468, 1997; Kren, et al., Proc. Natl. Acad. Sci. USA, 96,10349-10354,1999; Barlett, et al., Nat. Biotechnol, 18, 615-622, 2000; Rando, Proc. Natl. Acad. Sci. USA 97, 5363-5368, 2000; Santanna, et al, J. Invest. Dermatol, 111,1172-1177, 1998; Beetham, et al, Proc. Natl. Acad. Sci. USA 96, 8774-8778, 1999; Zhu, et al., Proc. Nati. Acad. Sci. USA 96, 8768-8773, 1999; Zhu, et al., Nat Biotechnol, 18, 555-558, 2000; Alexeev, et al., Nat Biotechnol, 16,1343-1346,1998; Alexeev, et al., Nat. Biotechnol, 18, 43-47, 2000) for recognition and repair of a target DNA sequence. Antisense and ribozyme oligonucleotide technologies are used to make changes (editing) in an RNA sequence. (Jones and Sullenger, Biotechnol,15, 902-905, 1997 and Sierakowska, et al, Proc. Natl. Acad. Sci. USA, 93, 12840-12844, 1996). Unlike gene replacement, where a therapeutic gene is transferred to the cell or affected organ (usually by virus-mediated gene transfer), therapeutic nucleic acid repair will produce predefined alterations in the DNA or RNA of eukaryotic cells. An advantage of gene repair over gene replacement is that the repaired gene is maintained, expressed, and regulated as the normal endogenous gene. Moreover, oligonucleotide repair will correct dominant or gain-of-function mutations that are not amenable to gene replacement strategy.

Various approaches have been attempted to improve the likelihood of a gene targeting event. One approach utilizes triple-helix-forming oligonucleotides coupled to a reactive chemical group (Havre, et al, Proc. Natl. Acad. Sci. USA, 90, 7879-83, 1993; Wang and Glazer, Science, 271, 802-805,1996), coupled to a single-stranded deoxyoligonucleotide or coupled to a double-stranded deoxyoligonucleotide. The deoxynucleotide coupled to the triple-helix-forming oligonucleotides contains a mismatch to the targeted base. (Chan et al., J. biol. Chem., 274, 11541-11548, 1999; Culver et al., Nat. Biotechnol,17, 989-993, 1999). The triple-helix-forming oligonucleotide recognizes the sequence surrounding a targeted base and the coupled reactive group or the coupled DNA elicits DNA repair and/or recombination, thereby resulting in an alteration of the sequence of the target nucleic acid. While the triple-helix-forming oligonucleotides are able to effect a change in the target DNA sequences, the frequency of inducing a change is approximately 1%. Moreover triple-helix-forming oligonucleotides are restricted in their target sequence, the target sequence must consist of homopurine or homopyrimidine stretches for the triplex formation.

Gene targeting techniques have also been applied to production of mice with targeted disruption of specific genes. Mice generated by these techniques have become invaluable tools to study the function of proteins in vivo(Muller, 1999). Current gene targeting techniques use homologous recombination in mouse embryonic stem (ES) cells to introduce site-specific modifications into the mouse genome. Using variations on this fundamental approach, it has become possible to produce mice with genetic alterations ranging from large deletions, to simple disruptions, to more subtle changes such as point mutations(Muller, 1999). As a testament to the power of these techniques, thousands of mice with disrupted genes have been generated since the technique was introduced in 1988(Mansour et al., Nature 336: 348-352,1988).

In 1996, Yoon, Kmiec and colleagues demonstrated that chimeric RNA-DNA oligonucleotides could introduce single base alterations into DNA, by what was called chimeroplasty(Yoon et al., Proc. Nat. Acad. Sci. 93: 2071-2076, 1996). Chimeroplasty was subsequently used to introduce single-nucleotide conversions into the genomic DNA in cultured lymphoblasts and hepatoma cells(Alexeev , V., Yoon, K., Nat. Biotechnol 16: 1343-1346, 1998;Cole-Strauss et al., Science 273:1386-1389, 1996;Kren et al., Hepatology 25: 1462-1468, 1997). Successful use of chimeroplasty to introduce single-nucleotide conversions in liver, skin and muscle cells in vivo has also been reported(Kren et al., Proc. Natl. Acad. Sci. 96: 10349-10354, 1999;Alexeev, V., and Yoon, K., Nat. Biotechnol 16: 1343-1346, 1998;Rando, T., A., et al., Proc. Natl., Acad. Sci. 97: 5363-5368, 2000). Attempts to use chimeroplasty for gene correction experiments have not always been successful. This may be due in part to difficulty in synthesizing and purifying double-stranded chimeric oligonucleotides. In addition, different cell types exhibit variation in the frequency of gene targeting events, perhaps due to different levels of the enzymes required for chimeroplasty(Santana et al., 1998).

The design of chimeric RNA-DNA oligonucleotides exploited the highly recombinogenic RNA-DNA hybrids and featured hairpin capped ends to avoid destruction by cellular helicases or exonucleases. The RNA-DNA oligonucleotides were shown to cause a site-specific chromosomal correction or mutation in tissue culture cells and in vivo. (Yoon, et al., Proc. Natl. Acad. SCi. USA 93, 2071-2076, 1996; Cole-Strauss, et al., Science, 273, 1386-1389, 1996; Kren, et al., Heptatogy, 25, 1462-1468, 1997; Kren, et al., Proc. Natl. Acad. Sci. USA, 96, 10349-10354, 1999; Barlett, et al., Nat. Biotechnol, 18, 615-622, 2000; Rando, Proc. Natl. Acad. Sci. USA 97, 5363-5368, 2000; Santanna, et al, J. Invest. Dermatol, 111,1172-1177, 1998; Beetham, et al, Proc. Natl. Acad. Sci. USA 96, 8774-8778, 1999; Zhu, et al., Proc. Natl. Acad. Sci. USA 96, 8768-8773, 1999; Zhu, et al., Nat Biotechnol, 18, 555-558, 2000; Alexeev, et al., Nat. Biotechnol, 16, 1343-1346, 1998; Alexeev, et al., Nat. Biotechnol, 18, 43-47, 2000). A permanent and stable gene correction by the RNA-DNA oligonucleotide was demonstrated by clonal analysis at the level of the genomic sequence, the protein, and by inducing a phenotypic change. (Alexeev and Yoon, Nat. Biotechnol, 16, 1343-1346, 1998). The RNA-DNA oligonucleotide might hold promise as a therapeutic method for the treatment of genetic diseases.

Oligodeoxynucleotides (ODN) have been widely used for inhibition of gene expression via an antisense mechanism. The sequence of the antisense oligonucleotide is complementary to the sequence of the mRNA and the antisense oligonucleotide has been shown to hybridize to the target mRNA. Suppression of gene expression was shown to occur by several mechanisms: cleavage and degradation of mRNA or hnRNA by RNase H, inhibition of ribosome binding to mRNA, or inhibition of translation.

Most recently, two groups have determined that single-stranded oligonucleotides, protected from degradation by phosphorothioate linkages or 2′O methyl RNA groups at both ends, can produce single base pair changes in DNA(Gamper, H. B., et al., Nucleic Acids Res. 23: 4332-4339, 2000; Igoucheva, O., et al., Gene Therapy 8: 391-399, 2001). These single-stranded oligonucleotides are easier to synthesize and purify than the original double-stranded chimeric RNA-DNA oligonucleotides, and produce gene conversion at similar frequencies to that reported for double-stranded molecules(Igoucheva, O., et al., Gene Therapy 8: 391-399, 2001). Oligodeoxynucleotides between 20-70 bases have been shown to cause DNA sequence changes in the yeast cyc1 gene (Moerschell, et al., Proc. Natl. Acad. Sci. USA, 95, 524-548, 1988; Yamamoto, et al., Genetics, 131, 811-819, 1992). The frequency of transformation ranged from 10⁻⁵ to 10⁻³, depending on the amount, length, and polarity of the oligodeoxynucleotide, as well as the genetic background of the recipient yeast.

Small-fragment homologous replacement strategy uses a 300-400 base single-stranded DNA to generate homologous replacement in mammalian cells, the efficacy of which is approximately 1%. (Gonez, et al, Hum. Mol. Genet 7, 1913-1919, 1998). It has been hypothesized that strand invasion of the single-stranded DNA into the targeted sequence results in pairing of the single-stranded DNA to either strand of the DNA target, similar to homologous recombination.

The present invention uses 25-61 nucleotide long oligonucleotides. These oligonucleotides are homologous to a target sequence, with the exception of a single mismatch to a targeted base in the targeted DNA. This short oligonucleotide is capable of a sequence-specific correction at the targeted base. The present invention exemplifies the efficacy of the invention using a mutant β-galactosidase and mutant green fluorescent protein (EGFP) gene. Correction of the mutation in the β-galactosidase and/or EGFP gene occurs in in vitro reactions using nuclear extracts, in episomes, and in the chromosome of mammalian cells (exemplified herein, but not meaning to limit, in CHO cells, ES cells and melanocytes). Thus, the methods of the present invention are useful for modifying a target gene in any cell, such as, but not limited to, mammalian cells (including but not limited to, bovine, ovine, porcine, equine, rodent and human), tissue culture cells, etc.

Development of a shuttle system wherein a mutant gene is inserted into a plasmid shuttle vector where the gene product is expressed in both mammalian and bacterial cells makes it possible to detect gene correction events in both types of cells. The β-galactosidase gene allows for a gene correction event to be determined by a simple color selection (blue or white) either by growing bacteria on X-Gal plates or by histochemical staining of mammalian cells. The β-galactosidase gene contains a single point mutation (G to A), resulting in the loss of enzymatic activity. (Igoucheva et al., Gene Ther. 6, 1960-1971, 1999). A short oligonucleotide directed to the correction of that point mutation caused a sequence-specific, length dependent, strand specific gene correction in mammalian cells.

Correction of a point mutation in mouse ES cells further exemplifies the efficacy of the present invention. The evidence available to date suggests that oligonucleotide-directed gene conversion requires mismatch repair machinery to operate(Santana, E., et al., J. Invest. Dermatol. 111: 1172-1177, 1998). This has been confirmed in cell-free systems(Cole-Strauss, A., et al., Nuc. Acid Res. 27: 1323-1330, 1999). Additional evidence suggests that homologous recombination may also be required(Igoucheva, O., et al., Gene Therapy 6: 1960-1971, 1999). Mouse ES cells are thus an attractive system in which to use oligonucleotides to produce subtle alterations in DNA, as they have active homologous recombination and mismatch repair activities(Ramirez-Solis, R., et al., Methods Enzymol. 2252: 855-878, 1997;Abuin, A. et al., Mol. Biol. Cell 20: 149-157, 2000). In addition, mice produced from ES cells result in a transgenic non-human animal line with specific single base changes. These transgenic animals are excellent models of genetic diseases. To date, there are no reports of the use of oligonucleotide-directed DNA alteration in mouse ES cells.

The present invention uses short deoxyoligonucleotides that are designed to effect a sequence-specific change in a target sequence, thereby generating a predefined alteration in the target sequence. This sequence-specific change is maintained in progeny cells. The present invention therefore solves a long sought need to develop a simple system to effect a genetic change, and to maintain this genetic change throughout the lifespan of the target cell.

### DESCRIPTION OF THE DRAWINGS

FIG. 1. Oligonucleotide sequences. The lower case indicates 2′-O-methyl RNA and the upper case indicates DNA. The mismatched base to the mutant β-galactosidase sequence is in bold letter.

FIG. 2. The relative frequencies of episomal gene correction by antisense (square) and sense (circle) oligonucleotides of different lengths. The number of blue stained cells ranged from 5˜2000 for each well containing 5×10⁴ cells, depending on the oligonucleotides used. For each set of transfection experiments, the relative frequency of each oligonucleotide was determined by dividing the number of blue cells found per well by the number found in the β-Gal Q (SEQ. ID. NO: 1) transfected cells. Standard deviation indicates the variation among at least five separate sets of episome transfections at 1 μM oligonucleotde concentration and 2 nM pCH110-G1651A plasmid.

FIG. 3. The histochemical staining of episome gene correction by antisense (left panel) and sense (right panel) oligonucleotides of different lengths. Each micrograph shows CHO-K1 cells in a 6-well plate and contains >600 cells per field (4). Panels A, C, E, and G depict CHO-K1 cells transfected with the antisense oligonucleotides β-Gal Q (SEQ. ID. NO: 1), W1 (SEQ. ID. NO: 2), X1 (SEQ. ID. NO: 3), Y1 (SEQ. ID. NO: 4), respectively at 1 μM and pCH110-G1651A plasmid at 2 nM (infra). Panels, B, D, F, H depict CHO-K1 cells transfected with sense oligonucleotides β-Gal R (SEQ. ID. NO: 5), W2 (SEQ. ID. NO: 6), X2 (SEQ. ID. NO: 7), Y2 (SEQ. ID. NO: 8), respectively at 1 μM and pCH110-G1651A plasmid at 2 nM.

FIG. 4. (A) Chromosomal gene correction is oligonucleotide dose-dependent. The number of blue cells was counted per well for clone 14 CHO-K 1 cells containing the mutant β-galactosidase in the chromosome: β-Gal W1 (SEQ. ID. NO: 2) (square) and β-Gal X1 (SEQ. ID. NO: 3) (circle). (B) Chromosomal gene correction is dependent on the length and polarity of the oligonucleotide. The number of blue stained cells ranged from 0˜100 for each well, depending on the oligonucleotide used, antisense (square) and sense (circle). For each set of transfection experiments the number of blue cells was counted per well for clone 14 CHO-K1 cells treated with in each oligonucleotide. The standard deviation indicates the variation among at least five separate sets of transfection experiments.

FIG. 5. Stability of the oligonucleotides. A trace amount of the ³²P end-labeled oligonucleotide was added to CHO-K1 cells and was isolated at various time points. Lanes 1, 2, and 3 indicates oligonucleotides isolated at 0, 6 and 24 h after transfection, respectively.

FIG. 6. D-loop formation promoted by nuclear proteins. Reaction containing ³²P-labeled β-Gal X1 (AS; SEQ. ID. NO: 3) or β-Gal X2 (S; SEQ. ID. NO:7) was incubated with homologous (h) and heterologous (ht) superhelical DNA, as described infra. D-loop formation was performed in the absence (lanes 1-4 ) or presence (lanes 5-10) of nuclear extracts. Except lanes 9 and 10 where the concentration of dsDNA was increased to 20 nM, all reactions were carried out with 2 nM of dsDNA and 84 nM oligonucleotide. The arrows indicate different forms of dsDNA: nicked circle (NC), linear (L), and superhelical (SC).

FIG. 7. Oligonucleotides sequences. Oligonucleotides used with different mutant reporter plasmids are indicated. For each reporter, the mutant DNA sequence is shown, and the mutant base indicated. The mutant codon for each reporter is underlined. Lower case letters indicate 2′-O-methyl RNA and upper case indicates DNA. Italicized lower case letters indicate phosphorothioate linked DNA. Symbol refers to the abbreviated oligonucleotide structure used in Table 2.

FIG. 8. Gene Conversion in CHO cells. CHO cells are transfected with mutant EGFP plasmids followed by control (A-C) or correcting oligonucleotides (D-I). Cells are viewed by phase (A,D,G) and fluorescence (B,E,H) microscopy, and then analyzed FACS (C,F,I). As can be seen, control oligonucleotides do not produce any glowing CHO cells (B,C). Transfection with correcting oligonucleotide G67 wt 5 (D-F, SEQ. ID. NO: 19), corrects the mutant EGFP plasmid in 0.57% of cells (E,F). Transfection with correcting oligonucleotide G67 wt 8 (G-I, SEQ. ID. NO: 22) corrects the mutant EGFP plasmid in 0.86% of cells (H,I).

FIG. 9 . Gene Conversion in ES cells-EGFP. ES cells are transfected with the Q177X mutant EGFP (SEQ. ID. NO: 22) plasmid combined with control or correcting oligonucleotides. Cells are viewed with phase (A,C,E) and fluorescence (B,D,F) microscopy. Fluorescent ES cells are detected following transfection with correcting oligonucleotides (D,F; SEQ. ID. NO: 26 and 27), but not following transfection with control oligonucleotide (B, SEQ. ID. NO: 28).

FIG. 10. Gene Conversion in ES cells-B-galactosidase. ES cells are transfected with mutant B-galactosidase plasmid combined with control (A,D) or correcting oligonucleotides (B,C,E,F). Cells are stained with X-Gal to detect B-galactosidase activity 48 hours after transfection. Blue ES cells are detected following transfection with the correcting oligonucleotide, indicating correction of the mutant B-galactosidase reporter gene in ES cells. No blue cells are seen following transfection with control oligonucleotides.

FIG. 11. Confirmation of specific gene conversion in ES cells. ES cells are transfected with mutant B-galactosidase plasmid combined with control or correcting oligonucleotides. Cells are harvested 48 hours after transfection, and Hirt DNA (episomal DNA) isolated. Hirt DNA is used to transform P90C cells. Blue colonies are observed only in DNA from transfections that include the correcting oligonucleotide β-gal wt 5 (SEQ. ID. NO: 34). Plasmid DNA isolated from these blue colonies demonstrates the specific A to G sequence correction at base 1651 of plasmid pCH110 (Blue Colony). In contrast, plasmid DNA isolated from white colonies contains the mutant base A at position 1651 (White Colony). No other sequence alterations are detected in the B-galactosidase coding regions of the isolated plasmids.

FIG. 12. Sequences of the ODN directed to the tyrosinase gene and the targeted sequences in tyrosinase. The target site is underlined (red) in the sequence. To protect the 3′ and 5′ end of the molecule, four residues of 2′-O-methyl uracil residues are incorporated at each end of ODN. DNA residues are capitalized and the 2′-O-methyl RNA residues are in lower case.

### DESCRIPTION OF THE INVENTION

Synthesis and Purification of Oligonucleotides

The oligonucleotides are synthesized on an Applied Biosystems (Foster City, Calif.) model ABI 392 RNA/DNA synthesizer, using a 1 micromole scale by standard phosphoramidite procedure. Chemicals used for the syntheses are purchased from Chem Gene (Cambridge, Mass.). The oligonucleotides are purified purified by denaturing electrophoresis on acrylamide gels as described (Yoon, et al., Proc. Natl. Acad. Sci. USA, 93, 2071-2076, 1996). All oligonucleotides used in these experiments are synthesized by the Nucleic Acid Facility at the University of Pennsylvania. Analytical gel electrophoresis of purified oligonucleotides demonstrats a single species of the correct size for each oligonucleotide used.

Plasmids

The mammalian shuttle vectors pCH110-G1651A and pcDNA3.1/Zeo/G1651A contain the lacZ gene with an inactivating G-to-A point mutation at position 1651. The plasmid pCH110-G1651A has been described previously. (Igoucheva, et al., Gene Ther., 6, 1960-1971, 1999). The pcDNA3.1/Zeo/G1651A plasmid was constructed by inserting a 3.7 kb fragment that contained the mutant lacZ gene into the BamHI-HindIII sites of pcDNA3.1/Zeo(+) plasmid (Invitrogen, Carlsbad, Calif.).

Cell Cultures

All media and fetal bovine serum (FBS) were from Gibco BRL and are supplemented with 2 mM L-glutamine. CHO-K1 cells (ATCC, Rockville, Md.) are maintained in F12 medium containing 10% heat-inactivated FBS. DT40 cells are grown in RPMI 1640 medium containing 10% heat-inactivated FBS, 1% chicken serum (Sigma, Saint Louis, Mo.) and 50RM 2-mercaptoethanol (Sigma, Saint Louis, Mo.). All cells are grown at 37° C. and 5% CO₂. AB2.2 ES cells (Stratagene) are cultured on mitomycin C innactivated STO feeder cells (ATCC), according to established protocols(Matise et al., 2000). TL-1(Labosky et al., 1997) and R1(Nagy et al., 1993) ES cells are grown on innactivated mouse embyronic fibroblasts.

Transfection and Selection

Several stable cell lines containing the integrated mutant LacZ gene are generated by transfection the pcDNA3.1/Zeo/G1651A construct into CHO-K 1 cells, selection for Zeo resistance, and cloning using a cloning cylinder. Selected clones are characterized at the DNA sequence level by RFLP analysis of genomic DNA. Expression of the mutant β-galactosidase is determined by Western Blot using a monoclonal anti-β-galactosidase antibody (Oncogene, Boston, Mass.), that recognized both wild and mutant protein.

For all transfections of ES cells, a ratio of 1 μg of DNA: 2.5 μg of Lipofectamine is used. In experiments where CM9 peptide is used, a ratio of 50 μg of peptide: 1 μg of DNA is used.

CHO cells are plated at a density of 5×10⁴ cells/well in 6-well plates 18 hours prior to transfection. For gene conversion experiments, CHO cells are transfected with 2 μg /well of reporter gene plasmids using lipofectamine plus CM9 peptide(Subramanian et al., 1999). Prior to transfection, 0.8 ml of fresh complete media is added to each well. The transfection mixtures, prepared in 0.2 ml of serum-free media (Optimem) are then added to the wells, and the plate centrifuged at 200 ×g for 5 minutes(Boussif et al., 1995). The transfection media is removed after 4-6 hours, and replaced with fresh media for 1 hour. Oligonucleotides are then transfected into the CHO cells overnight using the same method. The next day, the transfection media is removed, and replaced with fresh media. Cells are assayed for reporter gene activity 48 hours after starting the transfection.

ES cells are also transfected in 6 well plates with lipfectamine plus CM9 peptide using a total of 1 ml of media per well. Except where noted, 2 μg of reporter plasmid and 6 μg of oligonucleotide are used per well; this corresponds to a molar ratio of plasmid:oligo of approximately 1:750. For transfections, ES cells are trypsinized and then “panned” by plating them on gelatin-coated tissue culture dishes for 30-45 minutes to partially remove feeder cells. The panned ES cells are then pooled and counted. 2×10⁵ ES cells in 0.8 ml of media are then added to each well of a 6 well plate that contained feeder cells. The transfection mixtures, prepared in 0.2 ml of serum-free media (Optimem) are then added to the wells, and the plate centrifuged at 200 xg for 5 minutes. Tranfection media is replaced with fresh media after 4-6 hours. ES cells are assayed for reporter gene activity 48 hours after transfection.

Nuclear Extract Preparation and in vitro Analysis of Gene Conversion.

Nuclear extracts are prepared from DT40 cells as previously described. (Igoucheva, et al., Gene Ther., 6, 1960-1971, 1999). The standard in vitro reaction mixture contained 20 pM of supercoiled pCH110-G1651A DNA and 200 nM of ODN in a reaction buffer containing 30 mM Hepes (pH 7.8), 7 mM MgCl₂, 4 mM adenosine triphosphate (ATP), 200 μM each of cytosine triphosphate (CTP), guanosine triphosphate (GTP), uridine triphosphate (UTP), 100 μM each deoxy-ATP, deoxy-GTP, deoxy-CTP, deoxythymidine triphosphate (dTTP), 40 mM creatine phosphate, 100 μg/ml creatine phosphokinase and 15 mM sodium phosphate (pH 7.5). Incubation is carried out at 37° C. for 1 h. After reaction, the DNA is purified by phenol-chloroform extraction and precipitated with ethanol. Twenty percent of the recovered DNA is electroporated into E. coli strain P90C (setting 25 μF, 250 W, and 0.1 cm cuvette) and transformants are plated onto LB-dish containing 50 μg/ml of ampicillin and 100 μg/ml of X-Gal. The P90C [araΔ(lac proB)_{XIII}] has a deletion of the entire lac operon. (Cupples and Miller, Genetics, 120, 637-644, 1988). The frequency of correction is measured by dividing the number of blue colonies by the total number of colonies.

Nuclear extracts are prepared from ES cells grown in log phase. Extracts are assayed for gene-conversion activity using the β-galactosidase reporter plasmid (Igoucheva et al., 1999). Briefly, reporter plasmid and oligonucleotide are incubated in nuclear extract for 3 hours. The plasmid is extracted, and used to transform P90C bacteria. The bacteria are plated on LB agar plates containing X-Gal (100 μg/ml) and ampicillin (50 μg/ml). The number of blue colonies and total colonies are recorded.

Transfection and Histochemical Staining.

For episomal targeting experiments, 5×10⁴ cells are seeded per well in a 6-well plate 16-18 hours before transfection. Oligonucleotide (1 μM) and pCH110-G1651A plasmid (2 nM) are incubated with 15 μg LipofectAMINE™ (Gibco, Bethesda, Md.) in 1 ml OPTIMEM for 45 min and added to the cells. Cells are fed with 2 ml of a solution containing complete media 6 h later and stained 48 h after transfection. For histochemical staining, cells are washed three times with ice-cold PBS and fixed for 5 min in 1% glutaraldehyde at 4° C. After removal of fixation solution, cells are washed three times with PBS and then stained with X-Gal solution [5 mM K₃ Fe₃(CN)₆, 5 mM K₄ Fe₂(CN)₆, 1 mM MgCl₂ and 1 mM X-Gal (5-bromo-4-chloro-3-indolyl-β-D-galactoside)] in Hepes (pH 8.0) at 37° C. overnight. In order to prevent expression of endogenous mammalian β-galactosidase, histochemical staining is carried out at pH 8.0. Under these conditions only bacterial β-galactosidase is shown to be active. Blue cells are counted and averaged among ten fields (×10) by light microscopy.

For genomic targeting, 5×10⁴ cells are seeded per well in a 6-well plate 16-18 h before transfection. For lipofection, various amounts of ODN ranging 5˜15 μg, is diluted to 100 μl with OPTIMEM and added to 15-25 μg LipofectAMINE™ in final volume of 200 μl, made up with OPTIMEM. Complexes are allowed to form 45 min, after which time they are added to cells in a final volume of 1 ml, made up with OPTIMEM. Cells are fed with 2 ml of a solution containing complete media 6 h later and stained 48 h after transfection.

β-galactosidase activity in cultured ES cells is detected (supra). Blue cells with normal morphology are counted as positive. Dead or dying cells are not included in counts. Percent gene conversion in experiments with CHO cells is determined by dividing the number of blue cells by 1×10⁵, the number of cells expected to be present after one doubling of the cells plated in the wells.

Oligonucleotide Uptake Measurement and Stability.

Cellular uptake of oligonucleotide was measured in crude cellular lysate. (Santana, et al., J. Invest. Dermatol. 111: 1172-1177, 1998). The ³²P end-labeled oligonucleotide was transfected as described above. At various times of post-transfection, cells were extensively washed with PBS, followed by an acid wash in 1.5 M NaCl, pH 2.5 to strip off oligonucleotide bound to the plasma membrane. Cells were lysed in 1 ml Nonidet 40 solution (140 mM NaCl, 10 mM Tris-HCl, pH7.5, 1.5 mM MgCl₂, 0.5% Nonidet 40). After quantitating ³²P in each lysate, the samples were analyzed by 12% polyacrylamide gel electrophoresis containing 7 M urea followed by autoradiography. For gel electrophoresis, a crude lysate was further purified by phenol-chloroform extraction followed by desalting using G-25-spin column. (Boehringer, Indianapolis, Ind.).

RFLP Analysis and DNA Sequencing

Blue and white colonies generated by the in vitro reaction were isolated and subjected to PCR amplification by using two primers, 5′-GATGAAGCCAATATTGAAACC-3′(SEQ. ID. NO: 9) and 5′-CTGGTCTTCATCCACGCG-3′(SEQ. ID. NO: 10). The gene conversion was measured by HinfI digestion of the 300 bp PCR product. The Hinfl digestion of the PCR product from the white colony (AAATC) generates two fragments of 262 and 38 bp. In contrast, the PCR product from the blue colony (GAATC) generates 150, 112, and 38 bp fragments after Hinfl digestion. The plasmid DNA was subjected to a direct DNA sequencing by automatic DNA sequencer (ABI 373A, Applied Biosystems) using the primer, 5′-GATGAAGCCAATATTGAAACC-3′(SEQ. ID. NO: 9).

D-loop Formation Between ODN and Superhelical dsDNA

Twenty μg of nuclear proteins from DT40 cells were preincubated at room temperature for 5-10 min with 84 nM ³²P-labeled β-Gal X1 or βGal X2 in reaction mixture (20 μl) containing 20 mM Tris-HCl, pH 7.4, 1 mM DTT, 5 mM MgCl₂, 2 mM ATP, and 100μg/ml bovine serum albumin. After preincubation, superhelical pCH110-G1651A DNA was added at 2 nM or 20 nM into the reaction mixture and incubated at 37° C. for 5 min. As a control, an equal amount of p711 plasmid (Yoon, et al., Proc. Natl. Acad. Sci. USA, 93, 2071-2076, 1996) encoding the unrelated alkaline phosphatase is used as a heterologous DNA. The reaction is stopped by addition of 0.5% SDS and 2 mg/ml proteinase K at 37° C. for 15-20 min. The deproteinized products were analyzed on a 0.8% agarose gel in TBE buffer (45 mM Tris/45 mM boric acid/pH8.0/0.001 EDTA) containing 5 mM MgCl2 and run at 2.5-3.0 V/cm for 17-19 h at 4° C. The gel was dried and visualized by ethidium bromide staining. ³²P-labeled DNA was detected by autoradiography at −80° C. for 24-48 h.

Modifications

Modifications of the base, 3′ and/or 5′ end base modifications, backbone, and/or sugar moieties are incorporated into the oligonucleotides to increase the affinity of the oligonucleotides to the target sequence and to increase the oligonucleotides resistance against cellular nucleases. Hydrophobic modifications at the 5-position of pyrimidines, including, but not limited to, 2′-deoxyuridine, 5-fluoro-2′-deoxyuridine, 5-bromo-2′-deoxyuridine and 5-methyl-2′-deoxycytidine, will enhance the thermodynamic stability toward the target DNA. Further nucleobase modifications include, but are not limited to, other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

End (3′ and/or 5′) modifications include, but are not limited to, 2′-O-methyl bases, 3′ amine groups, phosphothioates, or any modified base that is nuclease resistant. These modifications are well known to those skilled in the art.

Various backbone modifications, such as phosphorothioates, phosphoramidites and methylphosphonates, and those with nonphosphate internucleotide bonds, such as carbonates, carbamates, siloxanes, sulfonamides and polyamide nucleic acid will increase the resistance to cellular nucleases.

In addition, sugar modifications, including but not limited to, 2′-O-methyl, a 2′-fluoro or a 2′-methoxyethoxy will increase the thermodynamic stability of the duplex, as well as the nuclease resistance. These modifications are incorporated and tested for effectiveness in gene conversion. The modifications incorporated into the oligonucleotide will not alter cellular functions that are responsible for biological activity, in this case recombination and repair activity.

Microscopy

Cells are examined and photographed in cultured dishes by light and fluorescence microscopy using a Nikon Diaphot inverted microscope.

FACS

For fluorescence activated cell sorting (FACS) analysis, cells are trypsinized, resuspended and PBS and kept on ice. FACS is performed in the Cell Sorting Core Facility at the University of Pennsylvania using a Becton Dickenson FACScan instrument. CellQuest software is used to acquire and analyze FACS results. The percent of gene conversion is determined by the number of live (gated) cells with fluorescence above background.

DNA Sequencing

Plasmid DNA is sequenced using Big Dye terminator cycle sequencing reagents (PE Biosystems). Reaction products are electrophoresed and analyzed on a 377 Automated DNA Sequencer in the Vision Research Core facility at the University of Pennsylvania.

Method of Administration

For administration, the oligonucleotides are dissolved in a physiologically-acceptable carrier, such as an aqueous solution or are incorporated within liposomes, and the carrier or liposomes are injected into the organism undergoing genetic manipulation, such as an animal requiring gene therapy or antiviral therapeutics. The preferred route of injection in mammals is intravenous. It is understood by those skilled in the art that oligonucleotides are taken up by cells and tissues in animals such as mice without special delivery methods, vehicles or solutions.

Administration of the oligonucleotides of the present invention is also performed locally to the area in need of treatment; this is achieved by, for example, and not by way of limitation, local infusion during surgery, topical application, or by means of an implant, the implant being of a porous, non-porous, or gelatinous material, including membranes, such as sialastic membranes, or fibers. Local infusion includes intradermal, subcutaneous, intranasal, and oral routes of administration. The oligonucleotides are administered by any convenient route, for example by infusion or bolus injection, by absorption through epithelial or mucocutaneous linings (e.g., oral mucosa, rectal and intestinal mucosa, etc.).

For in vitro research studies, a solution containing the oligonucleotides is added directly to a solution containing the DNA molecules of interest in accordance with methods well known to those skilled in the art.

The oligonucleotide can be made in a fashion so as to increase the stability of the oligomer under physiological conditions (supra). For example, changing the sugar/linkage backbone of the oligonucleotide can be applied to the oligomers herein described to increase the serum half-life of the oligonucleotide.

Methods of Use

If the target gene contains a mutation that is the cause of a genetic disorder, then the oligonucleotide is useful for mutagenic repair that will restore the DNA sequence of the target gene to normal. If the target gene is an oncogene causing unregulated proliferation, such as in a cancer cell, then the oligonucleotide is useful for causing a mutation that inactivates the gene and terminates or reduces the uncontrolled proliferation of the cell. The oligonucleotide is also a useful anti-cancer agent for activating a repressor gene that has lost its ability to repress proliferation. Furthermore, the oligonucleotide is useful as an antiviral agent when the oligonucleotide is specific for a portion of a viral genome necessary for proper proliferation or function of the virus.

The oligonucleotide is also used to generate a specific mutation in the target nucleic acid. For example, to generate a mutation in a cell line or in an animal which will provide a model to study the function of the gene product. This model is also used to test the efficacy of a potential therapeutic agent.

Stem cells are used in a body to replace cells that are lost by natural cell death, injury or disease. The present invention is also used for the correction and/or alteration of a gene in the pluripotent hematopoietic stem cells of humans in order to reconstitute all or part of the hematopoietic stem cell population of that individual. A stem cell is an undifferentiated cell capable of proliferation, self-maintenance, the production of a large number of differentiated functional progeny, and regenerating the tissue after injury. Stem cells of a particular tissue, for example the pancreas, are capable of differentiating into a variety of different pancreatic cell types (such as, but not limited to, pancreatic duct cells) when induced to proliferate. The method of the present invention is used to alter a target nucleic acid (e.g., gene) in a stem cell for the repopulation of a particular tissue(s).

The oligonucleotides herein described can be used alone or in combination with other agents. The two agents are administered in a fashion so that both agents are present within the cell or serum simultaneously.

Results

Gene Correction by oligonucleotides in Nuclear Extracts

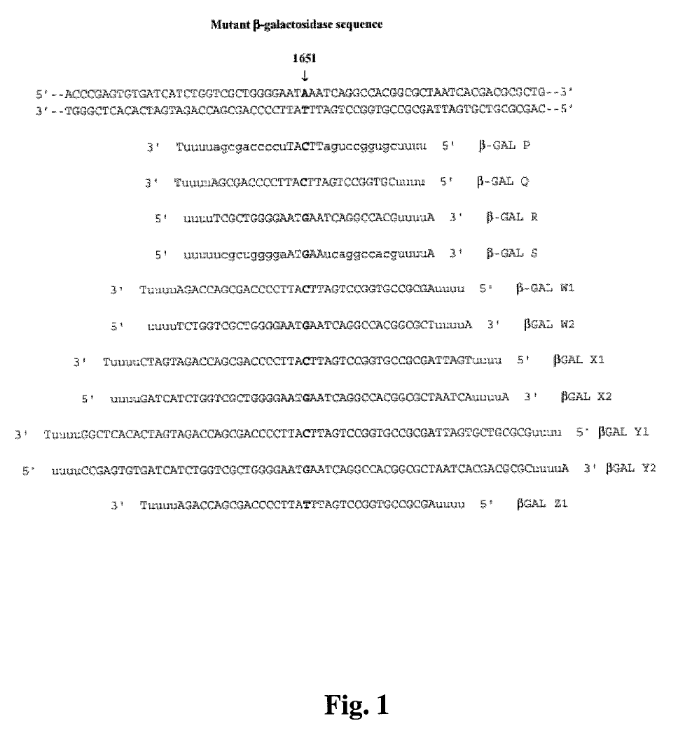

Using an in vitro reaction with nuclear extracts, several designs of oligonucleotide are compared for their gene correction activity. The parameters investigated are the length and polarity of oligonucleotide to the targeted sequence. Synthetic oligonucleotides used are shown in FIG. 1. Oligonucleotides are designed to restore enzymatic activity of the E. coli β-galactosidase by incorporation of a single mismatch into the targeted base. Control sequences included β-Gal Z1 (SEQ. ID. NO: 11) and β-Gal Z2 (SEQ. ID. NO: 12) (FIG. 1), which contained an identical sequence to the mutant. Increasing the length of oligonucleotide homology to the targeted sequence tested the importance of oligonucleotide length: 25, 35, 45 or 61 homologous bases are used.

Strand specificity is investigated by comparing the conversion frequencies between oligonucleotides in the antisense (β-Gal Q, β-Gal W1, β-Gal X1 and β-Gal Y1; SEQ. ID. NO: 1, 2, 3, and 4, respectively) and sense orientation (β-Gal R, β-Gal W2, β-Gal X2 and β-Gal Y2; SEQ. ID. NO: 5, 6, 7, and 8, respectively) (FIG. 1). At the same time, the correction activity of oligonucleotides of identical sequence, but containing 20 residues of RNA interrupted by five residues of DNA in the middle of the both the antisense (β-Gal P; SEQ. ID. NO: 13) and sense (β-Gal S; SEQ. ID. NO: 14) oligonucleotides, are analyzed. To protect the 3′ and 5′ ends of the oligonucleotides, four residues of 2′-O-methyl uracil residues are incorporated at each end of the oligonucleotide.

Initially, the ability of nuclear extracts from DT40 cells to catalyze the in vitro reaction between the supercoiled plasmid, pCH110-G1651A, and the oligonucleotide is measured. Reaction conditions are optimized by varying the amount of nuclear extract, the ratio of plasmid DNA to oligonucleotide, and the length of the reaction. DNA isolated from the in vitro reaction is transformed into E. coli P90C, which has a deletion of the entire lac operon. Thus, transformation of the plasmid containing a functional or a mutant β-galactosidase gene into P90C bacteria results in either a blue colony or a white colony, respectively, on X-Gal plates. The frequency of gene conversion is determined by dividing the number of bacterial colonies carrying a corrected lacZ gene (blue) by the total number of bacterial colonies (Table 1).

Using oligonucleotides of different lengths and polarity, the correction frequency ranged from between 2×10⁻⁴˜5×10⁻⁴ Although a slight increase in frequency is observed as the length of homology increased, this is statistically insignificant. In addition, there is no significant difference in gene correction frequency between the antisense and the sense oligonucleotides. Thus, neither the length nor the polarity of oligonucleotides appreciably affected the frequency of gene correction in the in vitro reaction.

A double-stranded oligonucleotide, composed of an equal molar ratio of β-Gal Q (SEQ. ID. NO: 1) and β-Gal R (SEQ. ID. NO: 5) (FIG. 1), shows a 4-fold lower gene correction frequency than either the sense or the antisense oligonucleotide alone. Furthermore, an oligonucleotide containing 20 RNA residues and five DNA residues of the identical sequence, β-Gal P (SEQ. ID. NO: 13) and β-Gal S (SEQ. ID. NO: 14) (FIG. 1) shows a frequency less than 10⁻⁵. Thus, a single-stranded oligonucleotide exhibits a higher gene correction frequency than a double-stranded DNA or an RNA oligonucleotide of the same sequence.

TABLE 1
Relationship between ssDNA length and gene
correction activity under in vitro reaction
conditions using mammalian nuclear extracts

| | | | |
|---|---|---|---|
| | | | Number of b |
| | Homology | | colonies/10⁵ t |
| ODN | length | Orientation | colonies^{a} |

| β-Gal P | 25 | AS | 0 |
|---|---|---|---|
| β-Gal Q | 25 | AS | 35 ± 3 |
| β-Gal R | 25 | S | 37 ± 4 |
| β-Gal S | 25 | S | 0 |
| β-Gal W1 | 35 | AS | 36 ± 33 |
| β-Gal W2 | 35 | S | 19 ± 2 |
| β-Gal X1 | 45 | AS | 41 × 33 |
| β-Gal X2 | 45 | S | 26 × 18 |
| β-Gal Y1 | 61 | AS | 47 × 17 |
| β-Gal Y2 | 61 | S | 25 × 3 |
| β-Gal Z1 | 25 | AS | 0 |
| β-Gal Z2 | 25 | S | 0 |
| ^{a}The frequency of gene correction is averaged among the results obtained from two separate in vitro reactions performed by least three different preparations of DT40 nuclear extracts. For consistency, each set of experiments used the same nuclear extracts for all oligonucleotides. One tenth of the DNA from the in vitro reaction is transformed into electro-competent P90C bacteria and plated into ten LB dishes containing 100 μg/ml | | | |
| # X-Gal and 50 μg/ml of ampicillin. The number of blue colonies is divided by the total number of colonies. | | | |

The control oligonucleotides containing the mutant sequence, β-Gal Z1 (SEQ. ID. NO: 11) and β-Gal Z2 (SEQ. ID. NO: 12) (FIG. 1), do not generate any blue colonies among the 10⁶ white colonies generated in five independent experiments, indicating a sequence-specific correction. This sequence conversion is not mediated by E. coli, as the gene correction event occurrs in mammalian cells, not in the bacteria (Igoucheva et al., Gene Ther., 6, 1960-1971, 1999). To verify the nature of the Lac+ phenotype revertants, RFLP analysis of DNA is performed on twenty blue colonies, followed by sequencing of the region surrounding the point mutation at position 1651 (FIG. 1). All twenty colonies exhibited a correction of AAA codon to GAA at position 1651 and no other DNA sequence changes were detected in the flanking regions.

Episomal Correction of a Point Mutation by Oligonucleotide

Oligonucleotide-based gene correction is analyzed in mammalian cells by targeting the episome. CHO-K1 cells are cotransfected with pCH110-G1651A plasmid and the oligonucleotide. Cells are stained 48 h after transfection with X-Gal solution for the presence of active β-galactosidase expression. When cells are transfected with the mutant plasmid DNA or cotransfected with plasmid and the oligonucleotides containing the mutant sequence, β-Gal Z1 (SEQ ID. NO: 11) or β-Gal Z2 (SEQ. ID. NO: 12) (FIG. 1), a complete absence of β-galactosidase enzymatic activity is observed. Similar to the in vitro reaction, no staining of the cells occurred when they are cotransfected with the RNA oligonucleotide, either β-Gal P (SEQ. ID. NO: 13) or β-Gal S (SEQ. ID. NO: 14) (FIG. 1).

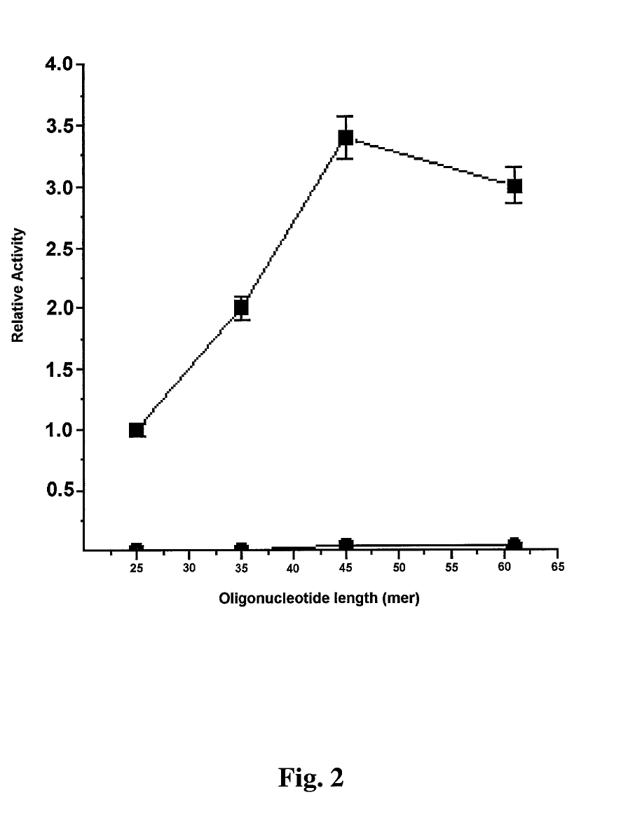

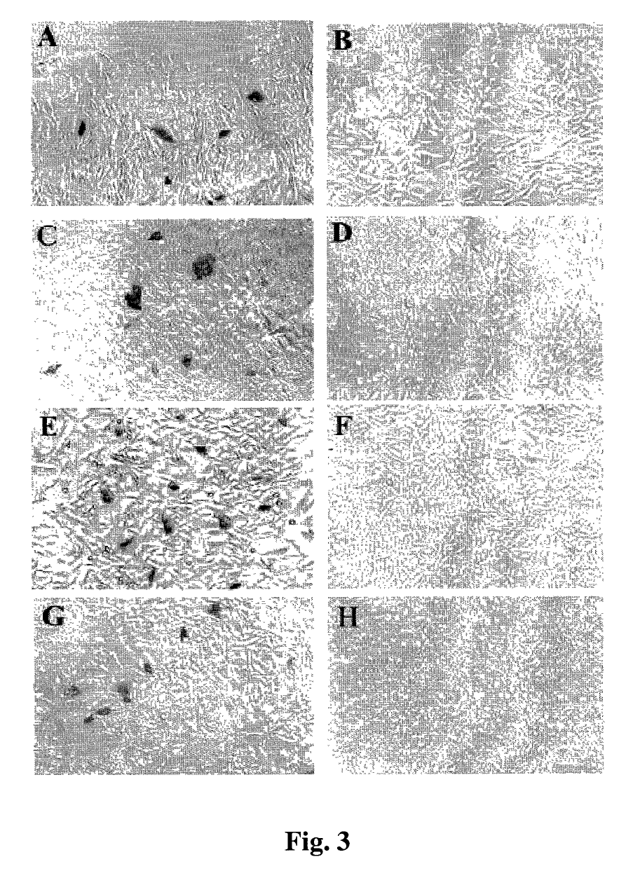

In contrast to the in vitro reaction, a homology-length dependent increase in the number of blue cells is observed (FIG. 2). Oligonucleotides with a homology length of 35 and 45 showed 2- and 4-fold higher gene correction frequency, respectively, in comparison to oligonucleotides with a homology length of 25. When the homology is extended to 61, a slight decrease in gene correction is observed.

The frequency of gene correction in the episome (0.5˜1%) is higher than that in the in vitro reaction (0.05%). In contrast to the in vitro reaction, two oligonucleotides with the same length, but opposite polarity, show strikingly different gene correction frequencies (FIGS. 2 and 3). An antisense oligonucleotide shows a much higher (>1000 fold) frequency of gene correction than a sense oligonucleotide. Thus, oligonucleotides of the present invention cause a sequence specific, homology-length dependent, and strand specific gene correction in the episome of mammalian cells.

Chromosomal Correction of a Point Mutation in CHO-K1 cells by oligonucleotide In order to investigate the feasibility of oligonucleotide gene correction at the chromosomal level, several stable cell lines are generated where the mutant LacZ gene is integrated. Ten independent clones are generated and subjected to Western Blot analysis using a monoclonal antibody, which recognizes both wild type and mutant β-galactosidase. Clone 14, which expresses the highest level of the mutant protein, is selected for a systematic study.

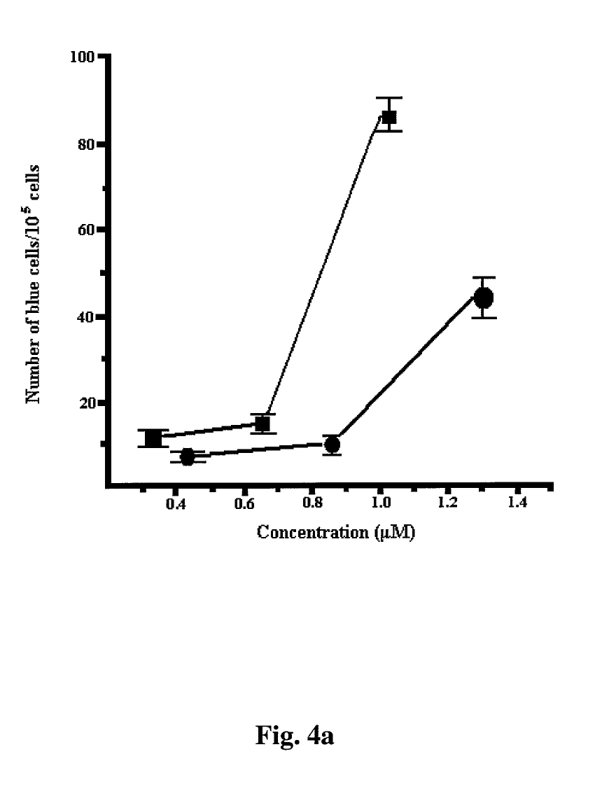

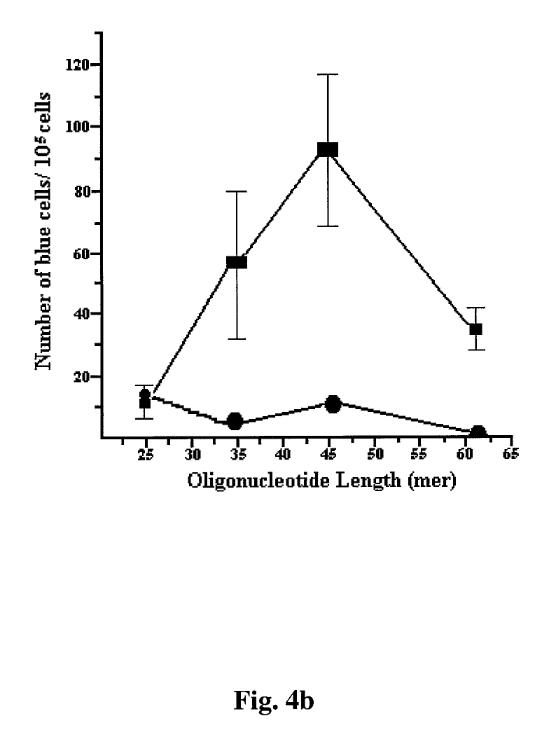

As the dose and the length of oligonucleotide increased, an increasing number of blue cells are detected (FIG. 4A). Similar to the episomal targeting, oligonucleotides with a homology length of 35 and 45 bases increases the frequency of correction to 2- and 4-fold, respectively, in comparison to the oligonucleotides with a homology length of 25 bases (FIG. 4B). Interestingly, the frequency decreased when the homology is extended to 61, indicating an optimum length of oligonucleotide exists for gene correction. The frequency of gene correction in the chromosome (˜0.1 %) is lower than that in the episome (0.5˜1%). A drastic difference in gene correction frequency between two oligonucleotides with the same length, but opposite polarity, is detected, similar to that detected in the episomal targeting (supra). An antisense oligonucleotide showed much higher frequency of gene correction than a sense oligonucleotide. Thus, oligonucleotides correct the chromosome of mammalian cells in a sequence-specific, homology-length dependent, and strand-polarity dependent manner.

Oligonucleotide Uptake and Stability

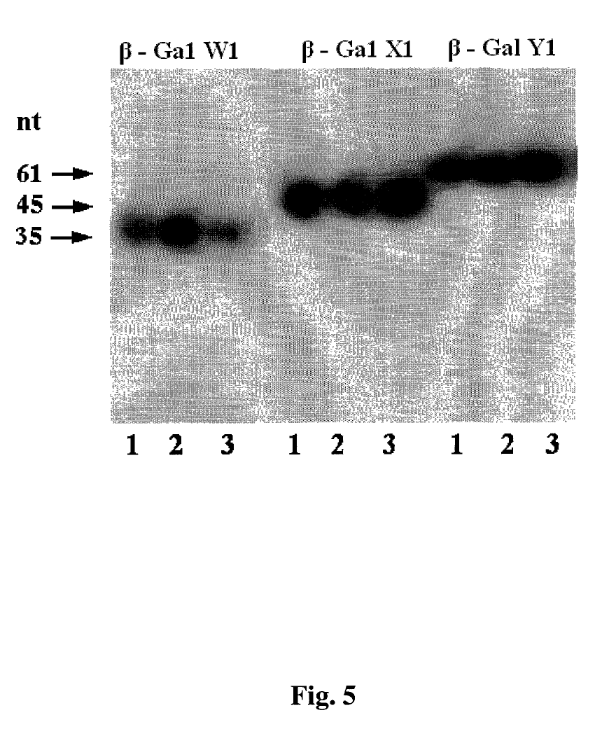

The length-dependent gene conversion frequency could result from two possibilities: either a longer oligonucleotide has a higher homologous recombination activity or a longer oligonucleotide is more nuclease resistant. In order to distinguish these possibilities, the stability of oligonucleotides in CHO-K1 cells is investigated. The cellular stability of the oligonucleotide is measured by transfection of a trace amount of ³²P end-labeled oligonucleotide into CHO-K1 cells. At various time intervals, 6 and 24 h after transfection, the oligonucleotide is isolated (supra) and analyzed by polyacrylamide gel electrophoresis, followed by autoradiography (FIG. 5). All tested oligonucleotides are stable and no detectable degradation is observed within 6h-24h. Thus, oligonucleotides are stable and remained as a monomer (i.e.: intact oligonucleotides) inside the cells.

Formation of D-loop Between Superhelical DNA and Oligonucleotide Using Nuclear Extracts

The initial step for gene correction by oligonucleotide would involve incorporation of oligonucleotide into the homologous duplex DNA, leading to a D-loop formation by homologous recombination. To investigate the ability of nuclear extracts to promote a sequence-specific D-loop formation, the ³²P-labeled β-Gal X1 (SEQ. ID. NO: 3) and X2 (SEQ. ID. NO: 7) are incubated with either homologous or heterologous superhelical DNA. Following incubation, proteins are inactivated by addition of proteinase K and SDS, and the reaction products are analyzed by 0.8% agarose gel electrophoresis.

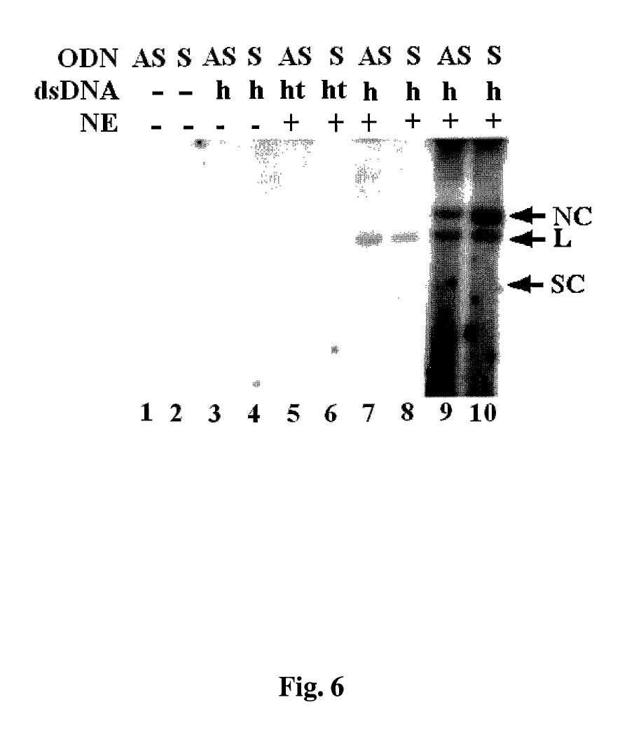

The incorporation of radioactivity into various forms of plasmid DNA is visualized by ethidium bromide staining and autoradiography of the same gel. The extent of D-loop formation is dependent on the amounts of nuclear proteins and plasmid DNA as well as an incubation time. Among three concentrations of nuclear proteins tested, 5, 20, and 50 μg, the optimal activity is observed at 20 μg. Higher concentration of homologous DNA promoted an increasing amount of D-loop formation (FIG. 6, compare lanes 7-10). A shorter time incubation of 5 minutes resulted in a higher amount of D-loop formation.

Assimilation of both sense and antisense oligonucleotides occurrs into the homologous DNA encoding the mutant β-galactosidase but not into the heterologous DNA encoding the alkaline phosphatase gene (FIG. 6, compare lanes 5,6 and 7,8). The sense and the antisense oligonucleotides show the same extent of D-loop formation, in agreement with the similar activity found in the in vitro reaction.

The incorporation of the oligonucleotide into both the nicked circle form and the linear form of dsDNA is observed, whereas no incorporation is observed in the superhelical DNA. Within 5 minutes of incubation with the nuclear extracts, the superhelical DNA is completely converted into the nicked circle form and the linear form, as detected by ethidium bromide staining. Thus, it is possible that the initial incorporation of the oligonucleotide into the superhelical DNA could have been converted to the D-loop formation between the oligonucleotide and the nicked circle form and the linear form.

It has been shown that the D-loop formation can result from nonenzymatic base pairing of a homologous single strand and superhelical DNA (Li, Z., et al, Proc. Natl. Acad. Sci. USA, 94, 11221-11226 (1997). When the D-loop formation is performed between the oligonucleotide and the homologous DNA in the absence of nuclear proteins, no incorporation of radioactivity is detected in any form of dsDNA (FIG. 6, lanes 3,4). This result indicates that D-loop formation is catalyzed by nuclear proteins and is dependent on the sequence homology and the amount of superhelical DNA.

Single Base DNA Alterations in Mouse ES Cells

EGFP and β-galactosidase Reporter Systems

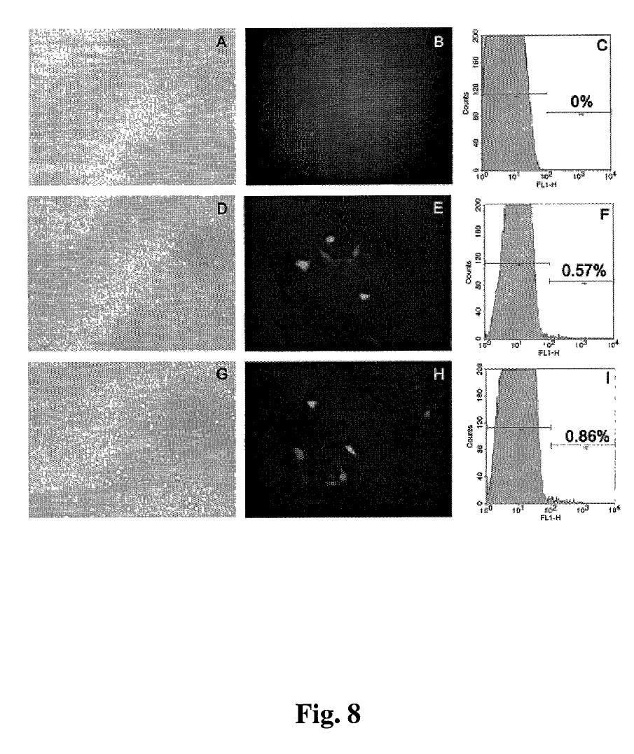

To provide simple assays for detecting single base changes produced by synthetic oligonucleotides in mammalian cells, two reporter gene systems are used. First, a reporter system is developed based on the enhanced version of green fluorescent protein (EGFP). EGFP protein autofluoresces, is easily detectable in and well-tolerated by mammalian cells(Yang et al., 1996;Cormack et al., 1996). Several missense and non-sense mutations are introduced into the EGFP gene to turn off protein fluorescence. These mutant EGFP's are then cloned into the eukaryotic expression plasmid pcDNA3.1 (Invitrogen), and transfected into Chinese Hamster Ovary (CHO) cells. Mutation G67R (nucleotide change G to C at base 202 in EGFP, SEQ. ID. NO: 17) in the EGFP chromophore and the non-sense mutation Q177X (nucleotide change C to T at base 532 in EGFP, SEQ. ID. NO: 24) produce no detectable fluorescence, as determined by both microscopy and fluorescence activated cell sorting (FACS) analysis (see FIG. 8 infra), and are chosen for further use.

The second reporter system (supra) is a β-galactosidase mutant. The β-galactosidase (supra) mutant used in the ES cells has an E523K mutation (G to A at nucleotide 1651 of plasmid pCH110) (Igoucheva et al., 1999). For both reporter systems, correction of the mutated nucleotide results in active proteins.

Synthetic Oligonucleotides

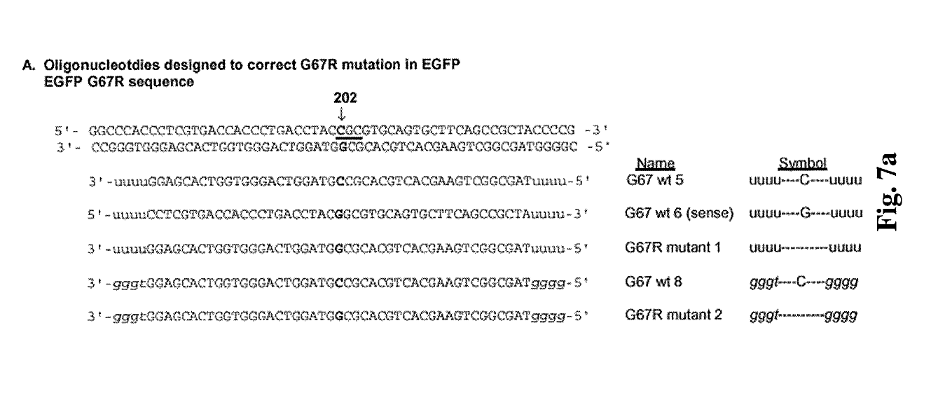

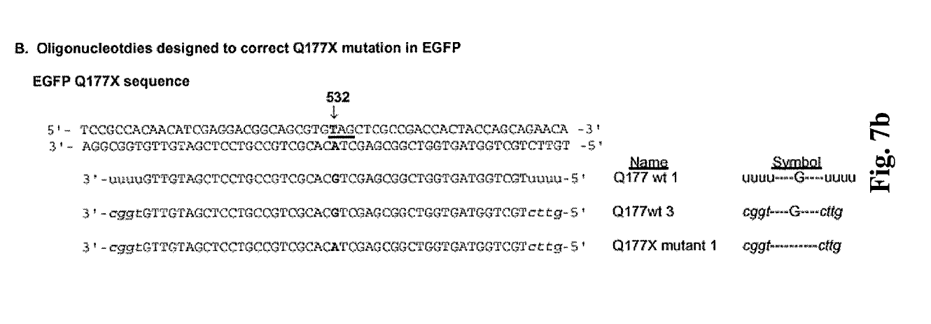

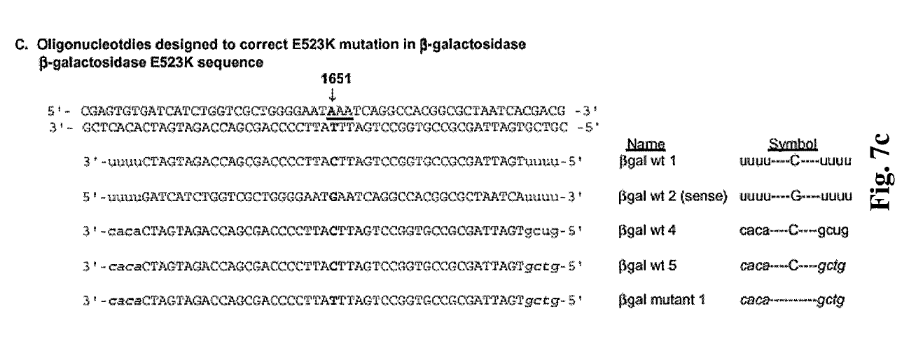

Oligonucleotides are designed to correct the G67R (SEQ. ID. NO: 17) and G177X (SEQ. ID. NO: 24) mutations in EGFP; successful use of the oligos produces fluorescent EGFP. Oligos are also prepared to correct the E523K mutation (SEQ. ID. NO: 29) in β-galactosidase (Igoucheva et al., 1999). The oligonucleotides initially tested are of the original double-stranded design(Yoon et al., 1996). Although these are found to be active, the present invention uses single-stranded oligonucleotides since they are more active, and easier to make and purify(Gamper et al., 2000;Igoucheva et a., 2001). All single-stranded oligonucleotides are in the antisense orientation, as this was found to provide increased conversion activity (supra and Igoucheva et al., 2001). Both 2′O methyl groups and phosphorothioate linkages at the 3′ and 5′ ends are used for nuclease protection(Gamper et al., 2000;Igoucheva et al., 2001). Structures of the oligonucleotides used in these experiments are shown in FIG. 7.

Gene Conversion in CHO Cells

To determine if the oligonucleotides of the present invention are active, CHO cells are transiently transfected with plasmids containing mutant EGFP or β-galactosidase genes, followed by a correcting oligonucleotide. Cells transfected with plasmid alone, or with plasmid plus control oligonucleotides without a mismatch (e.g. G67R mutant 1, SEQ. ID. NO: 21) are used in separate culture wells as controls. Two days after transfection, the cells are either stained with X-Gal to detect β-galactosidase activity or analyzed by fluorescence microscopy and FACS to detect EGFP. Results from a representative EGFP experiment are shown in FIG. 8. A summary of results from these experiments is presented in Table 2.

As can be seen in FIG. 8, control transfections using the G67R mutant version of EGFP (SEQ. ID. NO: 17) demonstrate no fluorescent cells (A,B,C). In contrast, when correcting oligonucleotides are used, fluorescent cells are detected in the culture plate (panels E,H) and by FACS analysis (panel F,I). Comparison of the M2 regions of the FACS histograms, indicating cells with fluorescent signal above background, showed 0.57% of cells are fluorescent following treatment with the G67 wt 5 oligonucleotide (SEQ. ID. NO: 19, panel F), and 0.86% fluorescent cells following treatment with oligonucleotide G67wt 8 (panel I, SEQ. ID. NO: 22). This amount of conversion is consistent with prior reports(Igoucheva et al., 2001). These results have been reproduced numerous times, with several different preparations of plasmid and oligonucleotides. Similar results are also obtained using the (SEQ. ID. NO:

24) mutant of EGFP and appropriate oligonucleotides.

TABLE 2
Oligonucleotide-directed gene conversion in
CHO cells.
βgal conversion - pCH110-g1651a plasmid

| Oligonucleotide | | #Blue cells | Calculated % Gene |
|---|---|---|---|
| Name | Structure | (mean) | conversion |
| βgal C | RDO | 11 | 0.022% |
| βgal wt 1 | uuuu----C----uuuu | 305 | 0.6% |
| (SEQ. ID. NO:31) | | | |
| βgal wt 2 | uuuu----G----uuuu | 115 | 0.22% |
| (SEQ. ID. NO:32) | | | |
| βgal wt 4 | caca----C----gctg | 369 | 0.74% |
| (SEQ. ID. NO:33) | | | |
| βgal wt 5 | caca----C----gctg | 511 | 1.02% |
| (SEQ. ID. NO:34) | | | |
| βgal mut 1 | caca---------gctg | 0 | — |
| (SEQ. ID. NO:35) | | | |

EGFP Conversion - pcDNA3-G67R-EGFP plasmid

| Oligonucleotide | | % Fluorescent cells |
|---|---|---|
| Name | Structure | by FACS (mean) |
| G67 wt 1 | RDO | <0.01% |
| G67 wt 5 (SEQ. ID. NO:19) | uuuu----C----uuuu | 0.44% |
| G67 wt 6 (SEQ. ID. NO:20) | uuuu----G----uuuu | 0.02% |
| G67 wt 8 (SEQ. ID. NO:22) | gggt----C----gggg | 0.67% |
| G67R mut 1 | gggt---------gggg | 0% |
| (SEQ. ID. NO:21) | | |

Similar results are also obtained using the mutant β-galactosidase reporter plasmid and correcting oligos. A summary of data from the β-galactosidase reporter experiments in CHO cells is shown in Table 2. As determined previously, antisense oligonucleotides are the most active in stimulating gene-conversion. Phosporothioate-protected oligonucleotides generated the highest rates of gene conversion (β-gal wt 5, SEQ. ID. NO: 34), even when compared to 2′OMe protected oligonucleotides with the same number of homologous bases (β-gal wt 4, SEQ. ID. NO: 33).

In vitro Correction by ES Cell Extract

It has been reported that the ability of different cell types to carry out oligonucleotide-directed gene conversion varies(Santana et al., 1998). This variability is thought to be due to the presence or absence of the enzymes needed to perform homologous recombination and/or mismatch repair. In order to determine if ES cells have the enzymatic machinery needed to carry out gene conversion, nuclear extracts of mouse ES cells are tested using an in vitro method(Igoucheva et al., 1999). Mutant β-galactosidase reporter plasmid and correcting or control oligos are incubated in nuclear extracts from several different cell types. Following this incubation, the plasmid DNA is extracted and electroporated into P90C bacteria, which lack the entire lac operon. The resulting number of blue colonies is recorded. As can be seen in Table 3, mouse ES cell extract is nearly as active as CHO extract at correcting the single base mutation in the reporter plasmid. In contrast, embryonic fibroblast feeder cells, on which ES cells must be cultured, have less activity. These data indicate that mouse ES cells do express the enzymes necessary for oligonucleotide-directed gene conversion.

TABLE 3
In vitro assay of chimeroplasty by nuclear extracts.

| Cell Type | # blue colonies/total colonies | % Gene Conversion |
|---|---|---|
| CHO cells | 103/5 × 10⁵ | 0.02% |
| Mouse ES cells | 26/3 × 10⁵ | 0.01% |
| Feeder Cells | 13/6 × 10⁵ | 0.002% |

ES Cell Transfection

DNA for gene targeting experiments is traditionally electroporated into ES cells. This requires many ES cells (10⁷) and large amounts of DNA (25μg). The present invention uses the cationic peptide nuclear localization signal M9 (CM9) in lipid-based transfections, which greatly enhances gene expression and improves oligonucleotide uptake in ES cells. Mouse ES cells lipofected with CM9 retain their pluripotency, and contribute to the germline. Therefore, CM9 peptide is used for the ES cell transfections. With CM9 peptide, 2 μg of wild-type pcDNA3-EGFP plasmid transfects 12-14% of 2×10⁵ ES cells. It is worth noting, however, that oligonucleotide and plasmid compete for ES cell transfection when combined. For example, when 2 μg of pcDNA3-EGFP plasmid are combined with 6 μg of oligonucleotide, only 0.97% of ES cells are transfected, as determined by FACS.

Single-base Conversions in ES Cells

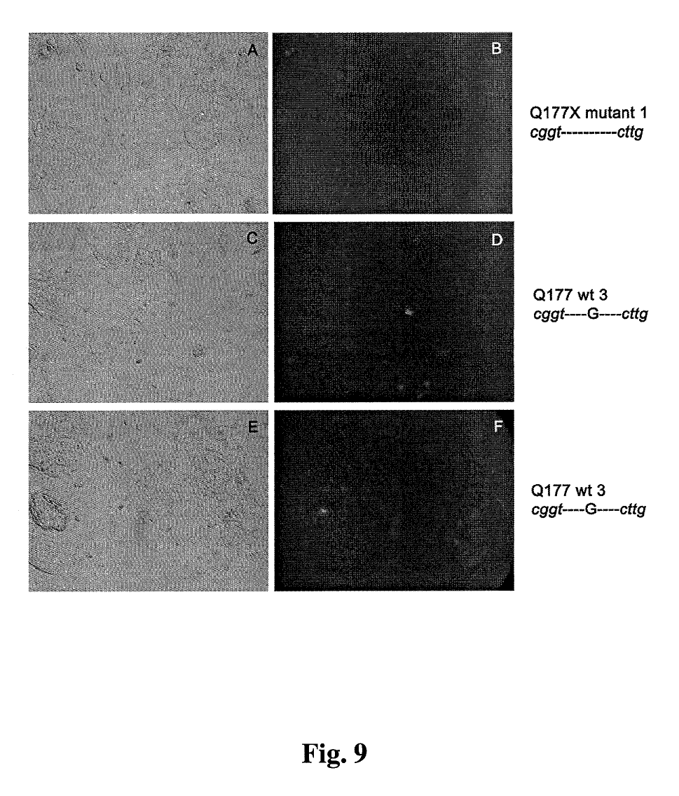

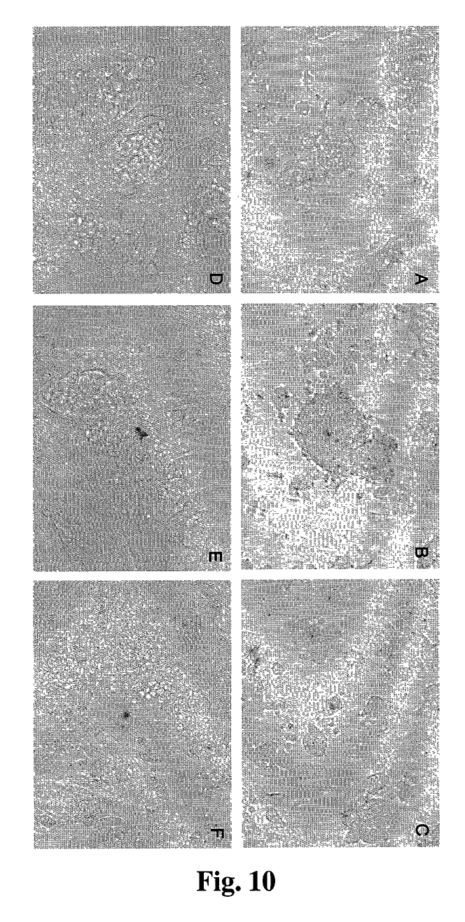

To test the ability of oligonucleotides to produce single-base alterations in ES cells, ES cells are transiently transfected with either EGFP or β-galactosidase reporter plasmid, combined with oligonuceotide. As can be seen in FIG. 9, correcting oligonucleotide Q177 wt 3 produced active EGFP in individual ES cells (panels C-F). No fluorescent ES cells are seen following transfection with plasmid alone, or with plasmid plus control oligonuceotide (panels A,B). As shown in FIG. 10, β-gal wt 5 oligonucleotide (SEQ. ID. NO: 34) produced active β-galactosidase in ES cells, as detected by X-Gal staining (panels B,C,E,F). Correction of the single base mutation in the β-galactosidase reporter plasmid in ES cells is detected repeatedly in 5 separate experiments, with 10-30 blue ES cells/well of the 6 well plate. ES cells treated with control oligonucleotide β-gal mutant 1 (SEQ. ID. NO: 35) showed no -galactosidase activity (panels A,B).

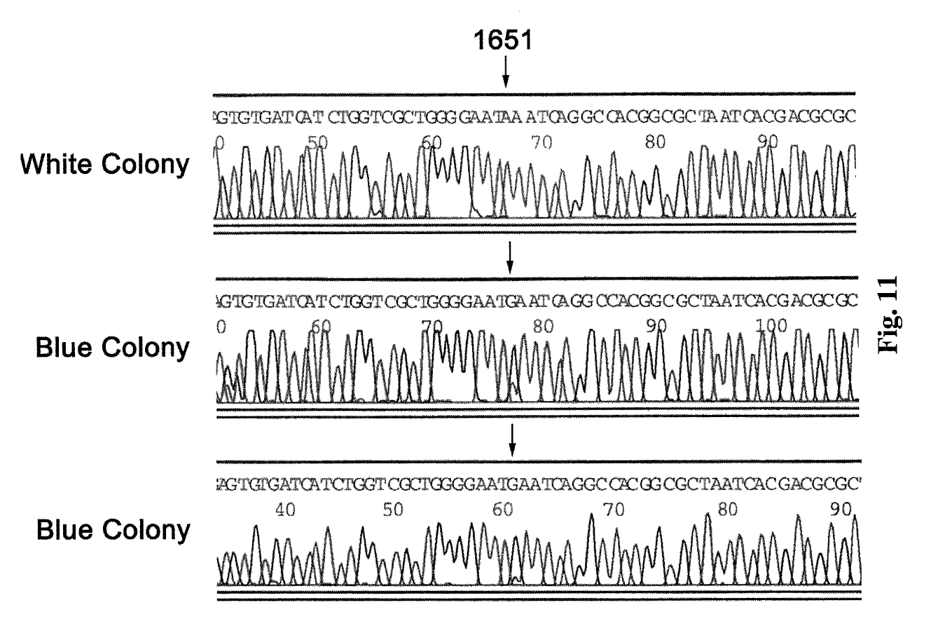

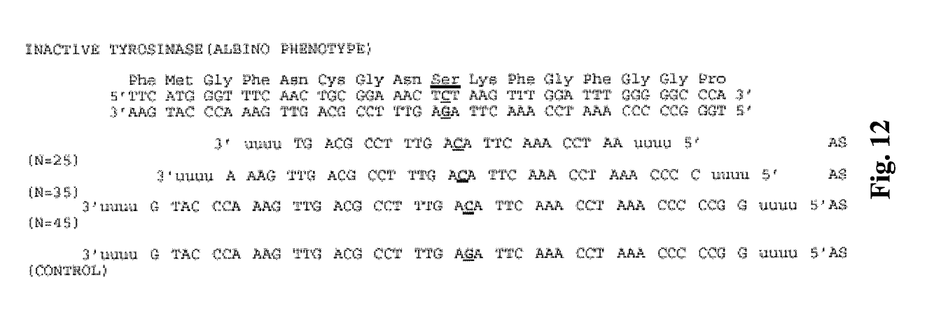

To verify that the blue cells observed following treatment with correcting oligonucleotide indicate β-galactosidase activity produced by specific correction of the G1651A mutation in the reporter plasmid, Hirt DNA is isolated from ES cells 48 hours after transfection with plasmid and oligonucleotide. The Hirt DNA is then used to transform P90C bacteria, which lack the entire lac operon. The transformed bacteria are plated on agar containing X-Gal, and the resulting number of blue colonies is counted. Hirt DNA isolated from ES cells transfected with correcting oligonucleotide produce approximately 0.7 blue colonies/1000 total colonies. In contrast, Hirt DNA isolated from ES cells treated with control oligonucleotide produce no blue colonies. Plasmid DNA is isolated from 5 blue colonies from the β-gal wt 5 (SEQ. ID. NO: 34) treated ES cell DNA. Sequencing of the entire β-galactosidase coding region in the plasmid DNA reveals specific base correction in the blue colonies, with no other base alterations noted (FIG. 11). Plasmid DNA is also isolated from 5 white colonies grown from the control-treated ES cell DNA. In all cases, the G1651A mutant β-galactosidase coding sequence is detected, without any other alterations (FIG. 11).

To verify that oligonucleotide-directed gene conversion can be used in multiple lines of ES cells, 2 additional ES cell lines are tested for this activity by transient transfection. TL1 and R1 mouse ES cells show levels of gene-correction activity similar to that observed for AB2.2 ES cells.

Gene Correction in Tyrosinase

All oligonucleotides (Table 4) are designed to restore the tyrosinase enzymatic activity by incorporation of a single mismatch (underlined) to the targeted base. Transfection of single-stranded ODN (Tyr N) in the antisense orientation and a homology length of 45 nucleotides is analyzed in melan c cells.

TABLE 4
The number of black cells among different
transfection experiment by Tyr N

| Passage Number | No of Melan | | | |
|---|---|---|---|---|
| of black cells | c cells | Tyr N | Liposome | No. |
| P16* | 20,000 | 2 μg | DMRIE | 8 |
| P17 | 20,000 | 2 μg | Superfectin | 20 |
| P17 | 20,000 | 2 μg | DMRIE | 20 |
| P18 | 20,000 | 2 μg | Superfectin | 7 |
| P20 | 20,000 | 2 μg | Superfectin | 10 |
| P21 | 20,000 | 2 μg | Superfectin | 8 |

*P denotes the passage number.

While this invention is described with a reference to specific embodiments, it is obvious to those of ordinary skill in the art that variations in these methods and compositions, such as the target gene and the cell to be treated, may be used and that it is intended that the invention may be practiced otherwise than as specifically described herein. Accordingly, this invention includes all modifications encompassed within the spirit and scope of the invention as defined by the claims.

Discussion

The present invention describes a relatively short deoxyoligonucleotide that causes a gene correction in episomal and chromosomal DNA in mammalian cells. A targeted gene correction of the E coli β-galactosidase gene containing a single point mutation by a chimeric RNA-DNA oligonucleotide (Igoucheva, et al., Gene Ther., 6, 1960-1971, 1999) has been previously shown to occur. Using this assay, oligonucleotides of the present invention are tested for their ability to alter the DNA sequence in mammalian cells at three different levels: in vitro reactions using nuclear extracts, in episomal DNA, and in chromosomal DNA. Suprisingly, a relatively short oligodeoxynucleotide by itself caused a gene correction in mammalian cells, similar to the chimeric RNA-DNA oligonucleotide.

Frequency of Gene Correction

The frequency of gene correction in the in vitro reaction is approximately 0.05% and is not dependent on the length or the polarity of the oligonucleotide. In contrast, the frequency of episomal DNA gene correction is highly dependent on the length and polarity of the oligonucleotide and ranges from 0.5% to 1% in CHO-K1 cells. Gene correction requires an optimum length of oligonucleotide, the oligonucleotode with a homology of 45 nucleotides shows the highest frequency of correction. Two oligonucleotides with the same length, but opposite polarity, show a drastic difference in gene correction frequency. An antisense oligonucleotide exhibits a much higher (>1000 fold) frequency of gene correction than a sense oligonucleotide.

Chromosomal gene correction shows a similar dependence on the length and polarity of oligonucleotide as does the oligonucleotide gene correction using episomal DNA, albeit at a lower frequency (approximately 0.1% in CHO-K1 cells). Thus, oligonucleotides cause a sequence-specific, a length dependent and a strand specific gene correction in both episomal DNA and chromosomal DNA of mammalian cells.

Episomal Gene Correction

The gene correction frequencies of episomal DNA were much higher than those observed in the in vitro reaction. This result indicated that some proteins were excluded or inactivated during the preparation of the nuclear extracts. A similar gene correction frequency was obtained using transient transfection of an RNA-DNA oligonucleotide into CHO-K1 cells, with approximately 1% gene correction. Similarly, an approximately 0.1% gene correction was obtained using an in vitro system consisting of nuclear extracts (Igoucheva, et al., Gene Ther., 6,1960-1971,1999).

Episomal gene correction frequency is also higher than gene correction in the chromosome. These differences could be due to the chromatin structure, which will limit the accessibility of the target chromosomal DNA. It is also possible that chromosomal recombination may be different from that of episomal recombination, which has been shown to occur by a nonconservative single-strand annealing mechanism (Lin, et al., Mol Cell Biol. 10,103-112, 1990; Segal and Carroll, Proc. Natl. Acad. Sci. USA, 91, 6064-6068, 1994; Rouet, et al., Proc. Natl. Acad. Sci. USA, 91, 6064-6068, 1994).

Recombinatorial Efficacy of Single-stranded Deoxyoligonucleotides

In all cases, a single-stranded deoxyoligonucleotide a shows higher frequency of gene correction than a double-stranded DNA or an RNA oligonucleotide of an identical sequence. Single-stranded DNA has a higher recombination activity due to its ability to invade the double-stranded target and its high affinity for recombinase. During a strand invasion, an RNA oligonucleotide can potentially make an RNA-DNA duplex, which is more active than a DNA duplex in homologous recombination by the RecA and Rec2 proteins (Kotani et al., Mol. Gen. Genet., 250, 626-634, 1996; Kmiec et al., Mol. Cell Biol. 14, 7163-7172, 1994). However, mismatch repair is less efficient in an RNA-DNA duplex than in a DNA-DNA duplex (Thaler et al., Proc. Natl. Acad. Sci. USA, 93, 1352-1356, 1996; Kamath-Loeb et a., Eur. J. Biochem., 250, 492-501,1997). A single-base correction in the target DNA is preferentially driven by the DNA-containing strand and not the RNA-containing strand of a chimeric RNA-DNA oligonucleotide (Igoucheva & Yoon, Gener Ther and Reg 1, 165-177, 2000). Therefore, a high frequency of gene correction by single-stranded oligonucleotide is attributed to a higher recombination in comparison to a double-stranded oligonucleotide. In addition, the DNA repair activity of an oligonucleotide results in a higher frequency of gene correction than does an RNA oligonucleotide.

The initial step for gene correction would involve a pairing of the oligonucleotide to the homologous DNA sequence by recombination. The nuclear proteins are found to catalyzed similar extents of the D-loop formation between both sense and antisense oligonucleotides and the homologous superhelical DNA. This result implies a pairing of the oligonucleotide to either strand of the homologous superhelical DNA and is in agreement with the similar in vitro gene correction frequency exhibited by both oligonucleotides.

Length of Dexyoligonucleotide for Gene Correction

In contrast to the in vitro reaction with nuclear extracts, the episomal or chromosomal gene correction is highly dependent on the length and polarity of the oligonucleotide. The D-loop formation by nuclear extracts also does not show an appreciable difference between the antisense and sense oligonucleotides of different lengths, indicating a good correlation between recombination and in vitro gene correction activity. Because oligonucleotides are quite stable and remained as a full-length monomer (i.e.: intact oligonucleotides) in mammalian cells, the increased gene correction frequency found in longer oligonucleotides is not likely to be caused by the stability of the longer oligonucleotides. Therefore, the optimal length of the oligonucleotide observed for the episomal and chromosomal gene corrections implies that other factors, such as the size and structure of a transiently open chromatin, play a role for initiating recombination.

Polarity of the Deoxyoligonucleotide for Gene Correction

A drastic difference in the gene correction frequency is observed when oligonucleotides of the same length but opposite polarity are targeted to both an episomal and chromosomal DNA. The antisense oligonucleotide exhibited greater than a 1000 fold higher frequency of gene correction than does the sense oligonucleotide. In contrast, the gene correction frequency of both the antisense and sense oligonucleotide is similar in the in vitro reaction. These results imply that transcription influences the extent of gene correction.

Transcription has been known to stimulate recombination and the preferential repair of the transcribed strand (Daniels & Lieber, Proc. Natl. Acad. Sci USA., 92, 5625-5629, 1995; Derr and Strathern, Nature, 361, 170-173, 1993; Mellon, et al, Cell, 51, 241-249, 1987; Bootsma & Hoeijmakers, Nature, 363,114-115, 1987). The preferential action of the antisense oligonucleotide is due to strand separation during transcription, which causes an opening of the chromatin, thereby allowing preferential accessibility to the non-transcribed strand. The RNA polymerase and accessory proteins occupy the transcribed strand, prohibiting the binding of an oligonucleotide. While both the antisense and sense oligonucleotides have an equal capacity for heteroduplex formation, only one strand will result in DNA sequence correction, due to the strand-specificity of the mismatch repair system (Modrich & Lahue, Annu Rev. Biochem, 65,101-133,1996).

In addition to hybridizing to the target DNA, the antisense oligonucleotide can hybridize to the mRNA and inhibit translation of the protein. This results in loss of protein activity. Therefore, the frequency by which the antisense oligonucleotide effects gene conversion, and thus gain of protein activity, would be higher than the frequency of gene conversion detected in the present invention, as inhibition of translation enhances the decrease in the protein expressed by the target gene.

Heteroduplex formation between the sense oligonucleotide and the transcribed strand can occur, but the sense oligonucleotide is excised and eliminated by the DNA repair system, which favors the resident strand over the invading strand resulting in no gene correction (Leung, et al., Proc. Natl. Acad. Sci USA, 94, 6851-6856, 1997). Yeast that were transformed with sense or antisense oligonucleotide were previously studied. The sense strand was shown to affect the target 50-100 fold more than the antisense oligonucleotide (Yamamoto, et al., Genetics, 131, 811-819, 1992). These results were independent of the transcription rate of the gene. It is unclear why such differences exist between yeast and mammalian cells.

Chan et al. showed that a bifunctional oligonucleotide, containing a triple helix domain and a donor fragment ranging 40-44 nucleotides homologous to the target DNA except one mismatch, corrected a point mutation in the episomal DNA approaching 1% in mammalian cells (Chan, et al., J. Bio.Chem. 274, 11541-11548, 1999). The bifunctional oligonucleotide showed a correction frequency 4-fold higher than either sense or antisense oligonucleotide. Regardless of the orientation, sense or antisense, the oligonucleotides showed similar gene conversion frequencies, but lower than the double-stranded oligonucleotide.

The gene correction frequency of the single-stranded oligonucleotides of the present invention is highly dependent on the polarity. More significantly, the gene correction frequency is higher than the double-stranded oligonucleotide. Differences between the present invention and that of Chan, et al. (supra) may be due to the different shuttle systems and assays. For example, the supF gene used in the bifunctional oligonucleotide was not transcribed, while the β-galactosidase gene of the present invention is transcribed in mammalian cells. Further, neither the length nor the polarity of the oligonucleotide appreciably affected the frequency of gene correction in the in vitro reaction of the present invention, where the β-galactosidase gene is not transcribed. Furthermore, Chan, et al. measured the frequency of gene conversion in the replicated episomal DNA in mammalian cells by transformation of the episome into bacteria. The present invention detects the frequency of gene conversion in the replicated episomal DNA in mammalian cells by direct detection of the corrected β-galactosidase gene expressed in those mammalian cells. These factors may contribute to the differences found between the two systems.

Relatively Short Deoxyoligonucleotides Effect a Sequence Specific Change in CHO Cells

While oligonucleotides have been widely used for the suppression of gene expression by an antisense effect, the present invention describes short deoxyoligonucleotides that cause a sequence-specific correction of both episomal and chromosomal DNA in mammalian cells. The present invention relates to 25-61 nucleotide long oligonucleotides that are homologous to a target sequence, with the exception of a single mismatch directed to a targeted base. Although the frequency of gene correction is relatively low, it is further improved by base, backbone, and sugar modifications that are incorporated into the oligonucleotides, thereby increasing the affinity of the oligonucleotide to the target sequence. In addition, the present invention includes base, backbone and sugar modifications to increase resistance to nuclease attack. The present invention uses relatively short deoxyoligonucleotides to effect a sequence-specific change in a target sequence in mammalian cells.

The Efficacy of Gene Correction in ES Cells

The efficacy of gene correction using the single-stranded deoxyoligonucleotides of the present invention are further used for the manipulation of DNA in ES cells, a powerful approach for generating animal models of disease. The data presented herein show the efficacy of synthetic oligonucletodies to create specific single-base alterations in DNA in mouse ES cells. The rate of gene conversion observed in ES cells is similar to that seen in other cell types (Igoucheva et al., 2001), thus targeting endogenous genes allows for specific alterations at a specified target nucleotide in endogenous genes. This ability to specifically alter a target nucleotide allows for the accurate generation of mouse models of inherited diseases, especially diseases involving dominant genes. Mice with engineered single-base mutations are also useful to test disease-specific therapeutic approaches to gene therapy or gene correction. Further, the present invention is useful for sequence specific alterations in a target nucleotide(s) in human stem cells, thereby allowing for the correction of mutations in stem cells from individual patients for therapeutic purposes.

The efficiency of gene conversion observed in CHO cells (supra) is similar to that reported by other investigators (Igoucheva et al., 2001). The single-stranded oligonucleotides of the present invention produce more base correction events than double-stranded chimeric oligonucleotides (supra). The antisense oligonucleotides are more effective at base correction than sense oligos (supra). Further, phosphorthioate(PS)-protected oligonucleotides produce more gene conversion than 2′O methyl uracil protected oligonucleotides (Table 2). This may be due in part to the longer region of homology in PS-protected oligos, as βgal wt 4 (SEQ. ID. NO: 33) with homologous 2′O methyl groups for protection is slightly more active than βgal wt with 2′O methyl uracil protection.

Frequencey of Gene Correction in ES Cells

The actual amount of base alteration observed in mouse ES cells is small, with only 10-30 out of 2×10⁵ cells, or 0.005-0.015%, demonstrating active reporter genes. A major reason for this, however, is the low efficiency of ES cell transfection. While lipofectamine and CM9 peptide can transfect 12-14% of ES cells with reporter plasmid alone, only 1% of ES cells are successfully transfected with plasmid when both reporter plasmid and oligonucleotide are combined for transfection. This 1% may also underestimate the number of cells that receive both plasmid and oligonucleotide. Thus, if all ES cells in culture are successfully transfected, the rate of base-correction is expected to be 100 times greater than the observed 0.5 to 1.5%. This is similar to the rate of base-correction observed in CHO cells (supra), and thus consistent with the in vitro conversion data which show a relatively similar activity of CHO and ES cell extracts (Table 3).

The conversion frequency of 0.5-1.5% observed in the oligonucleotide-directed single-base alterations in ES cells allows for the generation of mouse models of inherited diseases. One out of 200 ES cells treated with oligonucleotides harboring the desired mutation is readily detected by screening several 96-well plates of cloned ES cells. Several screening methods are available to detect single nucleotide mutations, such as, but not limited to, dot blotting, single-base extension, conformational methods such as DHPLC (denaturing high-performance liquid chromatography) or SSCP (single-strand conformational polymorphism), and direct sequencing of amplified DNA. As improvements in oligonucleotide structure continue, efficiency of mutation production will also increase.

Efficacy in Melanocytes

A point mutation in the tyrosinase gene in melanocytes is also corrected using the method of the present invention. The efficacy of gene correction in the melanocytes exemplifies, but does not limit, the applicability of the present invention for the treatment of skin diseases. The accessibility of the skin allows for therapeutics (i.e.: single-stranded deoxyoligonucleotides to correct a point mutation in a target cell) to be easily administered (including, but not limited to, topical application, subcutaneous injection, etc). Thus, diseases such as, but not limited to, psoriasis, epidermolysis bullose (EB) and albinism can be treated by altering a nucleotide in a target gene so as to either correct a point mutation so as to allow for the expression of an active protein or insert a mutation so as to inhibit the expression of that protein. For example, mutations in genes in the basal keratinocytes in the cutaneous basement membrane zone of the skin are implicated in causing the blistering skin disease EB. The method of the present invention allows for the generation of the appropriate gene conversion event, thereby treating the disease.

One Step Gene Correction

The use of oligonucleotides to introduce single-base mutations into endogenous genes in mouse ES cells provides an attractive approach to producing animal models of inherited diseases. The primary advantage of such a technique is the ability to introduce a specific single base change into a desired gene in a single step. In addition to introduction of specific mutations into known genes, it is also possible to use oligonucleotides to introduce nonsense mutations into genes, in either ES cells or other cell types (supra), as an alternative approach to disrupting gene expression.

What is claimed is:

1. A method of targeting and modifying, by mismatch repair, a pre-selected target nucleic acid, wherein a single-stranded oligonucleotide contains a mismatch to a targeted base in said pre-selected target nucleic acid, comprising:
a) administering said single-stranded oligonucleotide to a cell;
b) base pairing of said single-stranded oligonucleotide to said pre-selected target nucleic acid; and
c) incorporating said mismatch into said target nucleic acid.

2. The method of claim 1, wherein said single-stranded DNA oligonucleotide is complementary to either strand of said target nucleic acid with the exception of a nucleotide mismatch.

3. The method of claim 1, wherein said single-stranded DNA oligonucleotide comprises deoxynucleotide residues having a base modification, a 3′ and/or 5′end modification, a backbone modification or a sugar modification.

4. The method of claim 3, wherein said base modification comprises a modification of pyrimidines and/or purines, said modification selected from the group of consisting of: 5-fluoro-2′-deoxyuridine, 5-bromo-2′-deoxyuridine, 5-methyl-2′-deoxycytidine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

5. The method of claim 3, wherein said 3′ and/or 5′ end modification is at least one of the group of 2′-O-methyl bases, 3′ amine groups, phosphothioates, or any modified base which is nuclease resistant.

6. The method of claim 3, wherein said backbone modification is one of the group of phophorothioates, phosphoramidites, methylphosphonates, and modifications with nonphosphate internucleotide bonds, said nonphosphate internucleotide bonds selected from the group consisting of carbonates, carbamates, siloxanes, sulfonamides and polyamides.

7. The method of claim 3, wherein said sugar modifications are chosen from the group of 2′-O-methyl RNA, 2′-fluoro RNA and 2′-methoxyethoxy RNA.

8. A method of treating a genetic disorder, or other condition wherein an alteration of a target DNA sequence is desired, comprising:
a) administering to a mammal a therapeutically effective amount of an oligonucleotide;
b) base pairing of said oligonucleotide to said target DNA sequence, with the exception of a mismatch to a targeted base in said targeted DNA sequence; and
c) incorporating said mismatch into said targeted DNA sequence in a sequence-specific manner.

9. A method of targeting and modifying, by mismatch repair, a pre-selected target nucleic acid in a stem cell, wherein a single-stranded oligonucleotide contains a mismatch to a targeted base in said pre-selected target nucleic acid, comprising:
a) administering said single-stranded oligonucleotide to said stem cell;
b) base pairing of said single-stranded oligonucleotide to said pre-selected target nucleic acid; and
b) incorporating said mismatch into said target nucleic acid.

10.The method of claim 9, wherein said single-stranded DNA oligonucleotide is complementary to either strand of said target nucleic acid with the exception of a nucleotide mismatch.

11.The method of claim 9, wherein said single-stranded DNA oligonucleotide comprises deoxynucleotide residues having a base modification, a 3′ and/or 5′end modification, a backbone modification or a sugar modification.

12.The method of claim 11, wherein said base modification comprises a modification at the 5-position of pyrimidines, said modification selected from the group of consisting of: 5-fluoro-2′-deoxyuridine, 5-bromo-2′-deoxyuridine and 5-methyl-2′-deoxycytidine.

13.The method of claim 11, wherein said 3′ and/or 5′ end modification is at least one of the group of 2′-O-methyl bases, 3′ amine groups, phosphothioates, or any modified base which is nuclease resistant.

14.The method of claim 11, wherein said backbone modification is one of the group of phophorothioates, phosphoramidites, methylphosphonates, and modifications with nonphosphate internucleotide bonds, said nonphosphate internucleotide bonds selected from the group consisting of carbonates, carbamates, siloxanes, sulfonamides and polyamides.

15. The method of claim 11, wherein said sugar modifications are chosen from the group of 2′-O-methyl RNA, 2′-fluoro RNA and 2′-methoxyethoxy RNA.

16.A method of targeting and modifying, by mismatch repair, a pre-selected target nucleic acid in a skin cell, wherein a single-stranded oligonucleotide contains a mismatch to a targeted base in said pre-selected target nucleic acid, comprising:
a) administering said single-stranded oligonucleotide to said skin;
b) base pairing of said single-stranded oligonucleotide to said pre-selected target nucleic acid in a cell in said skin; and
b) incorporating said mismatch into said target nucleic acid.

17.The method of claim 16, wherein said single-stranded DNA oligonucleotide is complementary to either strand of said target nucleic acid with the exception of a nucleotide mismatch.

18.The method of claim 16, wherein said single-stranded DNA oligonucleotide comprises deoxynucleotide residues having a base modification, a 3′ and/or 5′end modification, a backbone modification or a sugar modification.

19.The method of claim 18, wherein said base modification comprises a modification at the 5-position of pyrimidines, said modification selected from the group of consisting of: 5-fluoro-2′-deoxyuridine, 5-bromo-2′-deoxyuridine and 5-methyl-2′-deoxycytidine.

20.The method of claim 18, wherein said 3′ and/or 5′ end modification is at least one of the group of 2′-O-methyl bases, 3′ amine groups, phosphothioates, or any modified base which is nuclease resistant.

21.The method of claim 18, wherein said backbone modification is one of the group of phophorothioates, phosphoramidites, methylphosphonates, and modifications with nonphosphate internucleotide bonds, said nonphosphate internucleotide bonds selected from the group consisting of carbonates, carbamates, siloxanes, sulfonamides and polyamides.

22. The method of claim 18, wherein said sugar modifications are chosen from the group of 2′-O-methyl RNA, 2′-fluoro RNA and 2′-methoxyethoxy RNA.
